(19) United States
(12) Patent Application Publication
(10) Pub. No.: US 2024/0254438 A1
(43) Pub. Date: Aug. 1, 2024

(54) MULTI-REGIONAL HUMAN NEURAL CIRCUITS IN ASSEMBLOIDS DERIVED FROM PLURIPOTENT STEM CELLS

(71) Applicant: The Board of Trustees of the Leland Stanford Junior University, Stanford, CA (US)

(72) Inventors: Sergiu P. Pasca, Menlo Park, CA (US); Ji-il Kim, Sunnyvale, CA (US); Yuki Miura, Menlo Park, CA (US)

(21) Appl. No.: 18/561,236

(22) PCT Filed: Jun. 21, 2022
(86) PCT No.: PCT/US2022/034341

§ 371 Date: Nov. 15, 2023

Related U.S. Application Data
(60) Provisional application No. 63/212,960, filed on Jun. 21, 2021.

(51) Int. Cl. C12N 5/079

(52) U.S. Cl. CPC C12N 5/0618; C12N 2500/38; C12N 2501/11; C12N 2501/115; C12N 2501/13; C12N 2501/155; C12N 2501/41; C12N 2503/02; C12N 2506/45; C12N 2513/00

(57) ABSTRACT

Functional human hCS-hStrS-hDiS and hCS-hDiS-hStrS-hMbS assembloids are generated by in vitro culture. Complete systems are assembled from component cultured cell systems, where each cultured cell system is designed to provide specific sets of neural cells, and which components are functionally integrated in the assembled spheroid.

### CROSS-REFERENCE TO RELATED APPLICATIONS

Pursuant to 35 U.S.C. § 119 (e), this application claims priority to the filing date of U.S. Provisional Patent Application Ser. No. 63/212,960 filed Jun. 21, 2021, the disclosure of which application is herein incorporated by reference.

### BACKGROUND OF THE INVENTION

Overactivity or hypoactivity of the circuit connecting the cortex, striatum, midbrain, and thalamus, as part of the direct loop pathway of the central nervous system, has been associated with a number of neuropsychiatric disorders, including autism spectrum disorder (ASD), Parkinson disease, schizophrenia and obsessive-compulsive disorder (OCD) (Kuo, H. Y. et al. eNeuro. 2019 Jun. 5; 6(3):ENEURO.0076-19.2019). However, the cellular and circuit mechanisms leading to disease in these conditions are still not fully understood. This is primarily due to limited access to intact, functional human brain tissue and preparation to probe and manipulate a cortico-striatal-midbrain-thalamic-cortical (CSMTC) loop circuit.

The CSMTC circuit controls movement behavior, habit formation, reward and other advanced functions of the central nervous system (Shepard. G. M. et al. Nat Rev Neurosci. 2013 April; 14(4):278-91). Parallel loops that start in different regions of the cerebral cortex, engage the striatum, midbrain and thalamus and project back onto regions of the cerebral cortex to modulate activity. For instance, the frontal loop involves pyramidal neurons in the frontal cortex providing excitatory glutamatergic input onto medium spiny neurons in the caudate, which in turn block the tonic activity of GABAergic neurons in the internal globus pallidus or substantia nigra pars reticulata (of the midbrain) and subsequently reduce inhibition on specific thalamic nuclei resulting in stimulation of the cerebral cortex. Aberrant activity at various levels of this loop has been associated with schizophrenia, ASD, epilepsy and other neurodevelopmental conditions.

There is great need for human models that can functionally integrate components of the corticospinal tract circuit, allowing screening for therapies that translate into treatments for patients.

Publications Birey et al. Nature 545: 54-59, 2017. Pasca et al. Functional cortical neurons and astrocytes from human pluripotent stem cells in 3D cultures. Nature Methods, 12: 671-78, 2015.

### SUMMARY OF THE INVENTION

Compositions and methods are provided for in vitro generation of a functional human cortico-striatal-midbrain-thalamic-cortical (CSMTC) loop circuit obtained by functionally integrating human assembloid brain-region specific organoids, which organoids may be generated at least in part from human pluripotent stem cells (hPSCs). Complete cortico-striatal-midbrain-thalamic-cortical spheroids (CSMTC) are assembled from component cultured cell systems, where each cultured cell system is designed to provide specific sets of neural cells, and which components are functionally integrated in the assembled spheroid (i.e. assembloids). Functionally integrated cells interact in a physiologically relevant manner.

Provided are methods to reconstruct, from parts derived from hPSC, a “direct” cortico-basal ganglia pathway in vitro. The methods generate 3D self-organizing cultures (also known organoids or spheroids) that resemble the human cerebral cortex (human cortical spheroids, hCS), the human diencephalon, including thalamus (human diencephalic spheroids, hDiS), human midbrain (human midbrain spheroids, hMbS) and human striatum (human striatal spheroids, hStrS), and then integrate them in various combinations (three-parts, four-parts) and different spatial arrangements (linear, circular) that allow formation of reciprocal connections and enable loop circuit-like properties. Assembly and live imaging with a calcium indicator of three-part assembloids created by integrating hCS, hStrS and hDiS has revealed synchronized activity. A four part assembloid from hCS, hDiS, hStrS, hMbS has been successfully maintained in culture for up to 3 months, and may be longer.

The spheroids may be arranged in any spatial arrangement deeded useful and may be particularly based on the number of distinct spheroids placed together in an assembloid. In some embodiments, when three part assembloids are used the spheroids in the assembloid may be arranged linearly. In some embodiments, when three part assembloids are used the spheroids in the assembloid may be arranged triangularly. In some embodiments, when three part assembloids are used the spheroids in the assembloid may be arranged circularly. In some embodiments, when four part assembloids are used the spheroids in the assembloid may be arranged linearly. In some embodiments, when four part assembloids are used the spheroids in the assembloid may be arranged squarely. In some embodiments, when four part assembloids are used the spheroids in the assembloid may be arranged circularly. In some embodiments, three and four part assembloids may be further supplemented with human subpallial spheroids (hSS) to enable cell migration.

The component systems of cortico-striatal-midbrain-thalamic-cortical spheroids include without limitation, human cortical spheroids (hCS); human striatum spheroids (hStrS); human midbrain spheroids (hMbS); and human diencephalic spheroids (hDiS). The component systems can be integrated into assembloids. The assembled spheroids (assembloids), i.e. hCS-hStrS-hDiS and hCS-hDiS-hStrS-hMbS provide unique opportunities for analysis and screening of agents that affect cortico-striatal-midbrain-thalamic-cortical circuits; including, without limitation, CNS injury, genetic mutations, infectious agents, therapeutic agents, nutritional factors, electrophysiological factors, and the like. Derivation of the assembled spheroids from pluripotent stem cells allows the development of patient-specific and disease-specific models.

In some embodiments, one or more such functional assembled spheroids, i.e. hCS-hStrS-hDiS and hCS-hDiS-hStrS-hMbS are provided, including without limitation a panel of such in vitro derived assembled spheroids, i.e. hCS-hStrS-hDiS and hCS-hDiS-hStrS-hMbS, where the panel includes spheroids generated from two or more genetically different cells. In some embodiments, the genome of each of component are the same or different. In some embodiments, a panel of such functional assembled spheroids are subjected to a plurality of candidate agents, or a plurality of doses of a candidate agent. Candidate agents include small molecules, i.e. drugs, genetic constructs that increase or decrease expression of an RNA of interest, infectious agents, electrical changes, and the like. In some embodiments a panel refers to functional assembled spheroids, or a method utilizing patient-specific functional assembled spheroids, from two or more distinct conditions, e.g. different genetic backgrounds, exposure to different drug treatments, exposure to pathogens, etc., and may be three or more, four or more, five or more, six or more, seven or more different conditions.

In some embodiments, methods are provided for determining the activity of a candidate agent on human cells present in the functional assembled spheroids, i.e. hCS-hStrS-hDiS and hCS-hDiS-hStrS-hMbS, the method comprising contacting the candidate agent with one or a panel of functional assembled spheroids. The cells present in the functional assembled spheroids optionally comprise at least one allele encoding a mutation associated with, or potentially associated with, a cortical disease, e.g. autism spectrum disorder (ASD), Parkinson disease, schizophrenia, Tourette syndrome, Huntington's disease, Timothy syndrome, Tuberous sclerosis, DiGeorge syndrome, Epilepsy and obsessive-compulsive disorder (OCD); and determining the effect of the agent on morphological, genetic or functional parameters, including without limitation gene expression profiling, migration assays, axonal growth and pathfinding assays, atomic force microscopy, super resolution microcopy, light-sheet microscopy, two-photon microscopy, patch clamping, cell death in neurodegenerative disorders, single cell gene expression (RNA-seq), calcium imaging with pharmacological screens, modulation of synaptogenesis and neuromuscular junctions, and the like.

Optionally, individual cell types of interest can be isolated from functional assembled spheroids, i.e. hCS-hStrS-hDiS and hCS-hDiS-hStrS-hMbS for various purposes. The cells are harvested at an appropriate stage of development, which may be determined based on the expression of markers and phenotypic characteristics of the desired cell type. Cultures may be empirically tested by immunostaining for the presence of the markers of interest, by morphological determination, etc. The cells are optionally enriched before or after the positive selection step by drug selection, panning, density gradient centrifugation, flow cytometry etc. In another embodiment, a negative selection is performed, where the selection is based on expression of one or more of markers found on hESCs or hiPSC, fibroblasts, epithelial cells, and the like. Selection may utilize panning methods, magnetic particle selection, particle sorter selection, fluorescent activated cell sorting (FACS) and the like.

The hStrS, hDiS and hMbS can be functionally integrated with human cerebral cortical spheroids (hCSs), which include pyramidal glutamatergic neurons of all cortical layers. The resulting assembled spheroid forms corticospinal projections and provides for functional integration of interneurons and cortical neurons. Using a combination of viral tracing, calcium imaging and electrophysiological methods, evidence is provided herein for the formation of an in vitro generated human cortico-striatal-midbrain-thalamic-cortical circuit, which provides useful modeling of injury, disease, and therapy.

These and other objects, advantages, and features of the invention will become apparent to those persons skilled in the art upon reading the details of the subject methods and compositions as more fully described below.

### BRIEF DESCRIPTION OF THE DRAWINGS

FIG. 1A-B. Schematic of the loop circuit and in vitro model.

FIG. 2A-K. Generation of thalamo-cortical assembloids.

FIG. 3A-C. Generation of cortico-striatal-thalamic-cortical (CSTC) loop assembloids.

FIG. 4A-B. Generation of cortico-striatal-midbrain-thalamic-cortical loop assembloids.

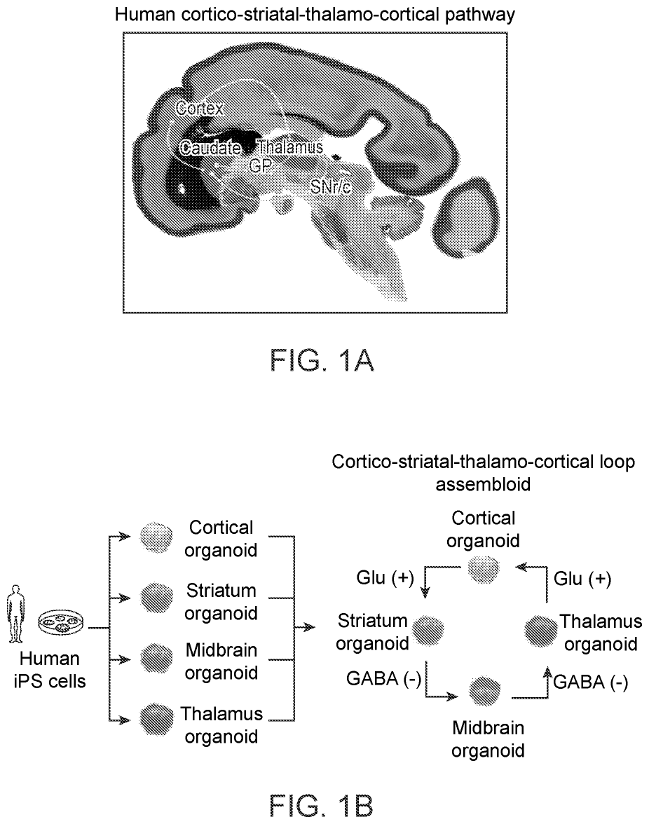

### DETAILED DESCRIPTION OF THE INVENTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a reprogramming factor polypeptide” includes a plurality of such polypeptides, and reference to “the induced pluripotent stem cells” includes reference to one or more induced pluripotent stem cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

By “pluripotency” and pluripotent stem cells it is meant that such cells have the ability to differentiate into all types of cells in an organism. The term “induced pluripotent stem cell” encompasses pluripotent cells, that, like embryonic stem cells (hESC), can be cultured over a long period of time while maintaining the ability to differentiate into all types of cells in an organism. hiPSC have a human hESC-like morphology, growing as flat colonies containing cells with large nucleo-cytoplasmic ratios, defined borders and prominent nuclei. In addition, hiPSC express pluripotency markers known by one of ordinary skill in the art, including but not limited to alkaline phosphatase, SSEA3, SSEA4, SOX2, OCT3/4, NANOG, TRA-1-60, TRA-1-81, etc. In addition, the hiPSC are capable of forming teratomas and are capable of forming or contributing to ectoderm, mesoderm, or endoderm tissues in a living organism.

Pluripotent stem cells may be obtained from patient or carrier cell samples, e.g. adipocytes, fibroblasts, keratinocytes, blood cells and the like. Various somatic cells find use as a source of hiPSCs; of particular interest are adipose-derived stem cells, fibroblasts, and the like. The use of hiPSCs from individuals of varying genotypes, particularly genotypes potentially associated with neurologic and neuromuscular disorders are of particular interest.

As used herein, “reprogramming factors” refers to one or more, i.e. a cocktail, of biologically active factors that act on a cell, thereby reprogramming a cell to multipotency or to pluripotency. Reprogramming factors may be provided to the cells, e.g. cells from an individual with a family history or genetic make-up of interest for heart disease such as fibroblasts, adipocytes, etc.; individually or as a single composition, that is, as a premixed composition, of reprogramming factors. The factors may be provided at the same molar ratio or at different molar ratios. The factors may be provided once or multiple times in the course of culturing the cells of the subject invention. In some embodiments the reprogramming factor is a transcription factor, including without limitation, OCT3/4; SOX2; KLF4; c-MYC; NANOG; and LIN-28.

Somatic cells are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector. The somatic cells may be fibroblasts, adipocytes, stromal cells, and the like, as known in the art. Somatic cells or hiPSC can be obtained from cell banks, from normal donors, from individuals having a neurologic or psychiatric disease of interest, etc.

Following induction of pluripotency, hiPSC are cultured according to any convenient method, e.g. on irradiated feeder cells and commercially available medium. The hiPSC can be dissociated from feeders by digesting with protease, e.g. dispase, preferably at a concentration and for a period of time sufficient to detach intact colonies of pluripotent stem cells from the layer of feeders. The spheroids can also be generated from hiPSC grown in feeder-free conditions, by dissociation into a single cell suspension and aggregation using various approaches, including centrifugation in plates, etc. The terms “spheroid” and “organoid” may be used interchangeably.

Genes may be introduced into the somatic cells or the hiPSC derived therefrom for a variety of purposes, e.g. to replace genes having a loss of function mutation, provide marker genes, etc. Alternatively, vectors are introduced that express antisense mRNA, siRNA, ribozymes, etc. thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as BCL-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

Disease-associated or disease-causing genotypes can be generated in healthy hiPSC through targeted genetic manipulation (CRISPR/CAS9, etc) or hiPSC can be derived from individual patients that carry a disease-related genotype or are diagnosed with a disease. Moreover, neural and neuromuscular diseases with less defined or without genetic components can be studied within the model system. A particular advantage of this method is the fact that edited hiPSC lines share the same genetic background as their corresponding, non-edited hiPSC lines. This reduces variability associated with line-line differences in genetic background. Conditions of neurodevelopmental and neuropsychiatric disorders and neural diseases that have strong genetic components or are directly caused by genetic or genomic alterations can be modeled with the systems of the invention.

Disease relevance. The effect of genetics, drugs, injury and pathogens on neurons is of particular interest, where efficacy and toxicity may rest in sophisticated analysis of neuronal projection, migratory and electrical interactions with neurons and non-neuronal cells, or the ability of neurons to form functional networks, rather than on simple viability assays. In addition, dysfunction in neural; pathways from the cortex to the striatum (cortico-striatal pathway), which may also involve neural pathways to/from the midbrain, is thought to contribute to severe neuropsychiatric disorders such as schizophrenia, obsessive-compulsive disorder, Tourette syndrome, Huntington's disease, Parkinson's disease and autism spectrum disorder (ASD) (Shepherd and Gordon, 2013). The discrepancy between the number of lead compounds in clinical development and approved drugs may partially be a result of the methods used to generate the leads and highlights the need for new technology to obtain more detailed and physiologically relevant information on cellular processes in normal and dysfunctional states.

Schizophrenia. The systems of the present invention provide unique opportunities to study schizophrenia. Schizophrenia is a chronic and severe mental disorder that affects an individual's behavior. The underlying cause of schizophrenia is not known, but the disorder has been associated with abnormal cortical dopamine signaling (Shepherd, 2014).

Obsessive-compulsive disorder (OCD) is a mental disorder in which a person feels the need to perform certain routines repeatedly. Cortico-striatal dysfunction is considered a major factor in OCD pathogenesis and functional imaging has shown increased or otherwise abnormal functional connectivity in the cortico-striatal pathways (Shepherd, 2014). Accordingly, the systems described here provide opportunities to further study OCD and develop potential therapeutic treatments.

Tourette syndrome is a neuropsychiatric movement disorder which is clinically characterized by the presence of vocal and motor tics. Whilst the underlying cause is still unclear, various studies support a hypothesis of a dysfunction in the cortico-striatal networks as a neurobiological substrate of tics. The systems described herein therefore allow for further study of the role of the cortico-striatal networks in Tourette syndrome and support the development of treatments.

Huntington's disease is a neurodegenerative disease characterized by the progressive loss of motor and cognitive function caused by degeneration of selected neuronal populations. Huntington's disease is mainly driven by a genetic defect on chromosome 4 that results in an expanded CAG repeat at the encoding site of huntingtin protein. The neurodegenerative process in Huntington's disease mainly affects the cortex and striatum. In the striatum primarily affects the medium spiny neurons that form part of the indirect pathway. The role of these pathways in Huntington's disease and potential therapeutic treatments can be further studied using the systems described herein.

Parkinson's disease is a progressive nervous system disorder that affects movement. It develops when neurons connecting the substantia nigra in the midbrain to the striatum degenerate, resulting in a loss of dopamine signaling. There is also evidence that there is a cortico-striatal aspect to the disease (Shepherd, 2014). Accordingly, the systems described herein provide an opportunity to further study the circuits underlying Parkinson's disease and to develop new treatments.

Autism spectrum disorder (ASD) is a developmental disorder characterized by defects in social-communication and the presence of repetitive/restricted behaviors, and is associated with defects in the cortico-striatal circuits. Various studies of ASD-associated genes have demonstrated cortico-striatal involvement. Mutations in SHANK3, a postsynaptic scaffolding protein expressed in medium spiny neurons, cause the ASD-related 22q13.3 deletion syndrome, also known as Phelan-McDermid syndrome.

Timothy syndrome (TS) is characterized by multiorgan dysfunction, including severe arrhythmias, webbing of fingers and toes, congenital heart disease, immune deficiency, intermittent hypoglycemia, cognitive abnormalities, epilepsy and ASD. There are two recognized types of Timothy syndrome, classical (type-1) and atypical (type-2). They are both caused by mutations in CACNA1C, the gene encoding the calcium channel Caᵥ1.2 α subunit. Timothy syndrome mutations in CACNA1C cause delayed channel closing, thus increased intracellular calcium. These mutations are in exon 8 (atypical form) and exon 8a (classical form), an alternatively spliced exon. Exon 8a is highly expressed in the heart, brain, gastrointestinal system, lungs, immune system, and smooth muscle. Exon 8 is also expressed in these regions and its level is roughly five-fold higher than exon 8a expression.

Tuberous sclerosis (TS) is a neurocutaneous syndrome that occurs in 1 of 6000 children; 85% of cases involve mutations in the TSC1 gene (9q34), which controls the production of hamartin, or the TSC2 gene (16p13.3), which controls the production of tuberin. These proteins act as growth suppressors. If either parent has the disorder, children have a 50% risk of having it. However, new mutations account for two thirds of cases. Central nervous system (CNS) tubers interrupt neural circuits, causing developmental delay and cognitive impairment and may cause seizures, including infantile spasms. Sometimes the tubers grow and obstruct flow of cerebrospinal fluid from the lateral ventricles, causing unilateral hydrocephalus. Sometimes tubers undergo malignant degeneration into gliomas, particularly subependymal giant cell astrocytomas (SEGAs).

22q11.2 deletion syndrome (also known as DiGeorge syndrome or velocardiofacial syndrome) is a primary immunodeficiency disorder that involves T cell defects. It results from gene deletions in the DiGeorge chromosomal region at 22q11.2, which cause dysembryogenesis of structures that develop from pharyngeal pouches during the 8th week of gestation. Most cases are sporadic; boys and girls are equally affected. Inheritance is autosomal dominant. Children with DiGeorge syndrome have a specific profile in neuropsychological tests. They usually have a below-borderline normal IQ, with most individuals having higher scores in the verbal than the nonverbal domains. Some are able to attend main-stream schools, while others are home-schooled or in special classes. The severity of hypocalcemia early in childhood is associated with autism-like behavioral difficulties Adults with DiGeorge syndrome are a specifically high-risk group for developing schizophrenia. About 30% have at least one episode of psychosis and about a quarter develop schizophrenia by adulthood. Individuals with DiGeorge syndrome also have a higher risk of developing early onset Parkinson's disease (PD).

Epilepsy is a group of non-communicable neurological disorders characterized by recurrent epileptic seizures. Epileptic seizures can vary from brief and nearly undetectable periods to long periods of vigorous shaking due to abnormal electrical activity in the brain. These episodes can result in physical injuries, either directly such as broken bones or through causing accidents. In epilepsy, seizures tend to recur and may have no immediate underlying cause.https://en.wikipedia.org/wiki/Epilepsy-cite_note-NEJM2003-10 Isolated seizures that are provoked by a specific cause such as poisoning are not deemed to represent epilepsy.https://en.wikipedia.org/wiki/Epilepsy-cite_note-Fisher2005-13 The underlying mechanism of epileptic seizures is excessive and abnormal neuronal activity in the cortex of the brainhttps://en.wikipedia.org/wiki/Epilepsy-cite_note-Fisher2005-13 which can be observed in the electroencephalogram (EEG) of an individual. The reason this occurs in most cases of epilepsy is unknown (idiopathic); some cases occur as the result of brain injury, stroke, brain tumors, infections of the brain, or birth defects through a process known as epileptogenesis. Known genetic mutations are directly linked to a small proportion of case.

### Cells and Structures

The adult cerebral cortex contains two main classes of neurons: glutamatergic cortical neurons (also known as pyramidal cells) and GABAergic interneurons. Pyramidal cells are generated in the pallium—the roof of the telencephalon (dorsal forebrain)—and reach their final position by radial migration. In contrast, cortical interneurons are born in the subpallium—the base of telencephalon (ventral forebrain)—and reach the cerebral cortex through a long tangential migration.

The layers of the cerebral cortex are generated in an “inside-out” sequence, with deep layers being generated first and superficial layer neurons being generated last. In parallel to this process, GABAergic interneurons migrate to the cortical plate, where they disperse tangentially via highly stereotyped routes in the marginal zone (MZ), subplate (SP), and lower intermediate zone/subventricular zone (IZ/SVZ). Interneurons then switch from tangential to radial migration to adopt their final laminar position in the cerebral cortex.

The movement of cortical interneurons is saltatory. First, the cell extends a leading process. Second, the nucleus translocates towards the leading process, a step referred to as nucleokinesis and leads to the net movement of the cell.

The translocation of the nucleus into the leading process is the mechanism that best defines this type of saltatory neuronal migration. First, a cytoplasmic swelling forms in the leading process, immediately proximal to the nucleus. The centrosome, which is normally positioned in front of the nucleus, moves into this swelling. The centrosome is accompanied by additional organelles, including the Golgi apparatus, mitochondria, and the rough endoplasmic reticulum. Second, the nucleus follows the centrosome. These two steps are repeated producing the typical saltatory movement of migrating neurons.

Tangentially migrating neurons do not always follow radial glial fibers. In general, tangentially migrating cells can move in clusters or individually. Cellular interactions also differ depending on the nature of the substrate. They can be homotypic, when interactions occur between cells of the same class, or heterotypic, when migrating cells rely on the contact with other cell types for their migration or their substrates. In the most common scenario, neurons respond to cues present in the extracellular matrix or in the surface of other cells to achieve directional migration.

GABAergic interneurons are inhibitory neurons of the nervous system that play a vital role in neural circuitry and activity. They are so named due to their release of the neurotransmitter gamma-aminobutyric acid (GABA). An interneuron is a specialized type of neuron whose primary role is to modulate the activity of other neurons in a neural network.

There are interneuron subtypes categorized based on the surface markers they express, including parvalbumin (PV)-expressing interneurons, somatostatin (SST)-expressing interneurons, VIP-expressing, serotonin receptor 5HT3a (5HT3aR)-expressing interneurons, etc. Although these interneurons are localized in their respective layers of the cerebral cortex, they are generated in various subpallial locations.

Morphologically speaking, cortical interneurons may be described with regard to their soma, dendrites, axons, and the synaptic connections they make. Molecular features include transcription factors, neuropeptides, calcium-binding proteins, and receptors these interneurons express, among many others. Physiological characteristics include firing pattern, action potential measurements, passive or subthreshold parameters, and postsynaptic responses, to name a few.

The PV interneuron group represents approximately 40% of the GABAergic cortical interneuron population. This population of interneurons possesses a fast-spiking pattern, and fire sustained high-frequency trains of brief action potentials. Additionally, these interneurons possess the lowest input resistance and the fastest membrane time constant of all interneurons. Two types of PV-interneurons make up the PV interneuron group: basket cells, which make synapses at the soma and proximal dendrite of target neurons, and usually have multipolar morphology and chandelier cells, which target the axon initial segment of pyramidal neurons.

The SST-expressing interneuron group is the second-largest interneuron group. SST-positive interneurons are known as Martinotti cells, and possess ascending axons that arborize layer I and establish synapses onto the dendritic tufts of pyramidal neurons. Martinotti cells are found throughout cortical layers II-VI, but are most abundant in layer V. These interneurons function by exhibiting a regular adapting firing pattern but also may initially fire bursts of two or more spikes on slow depolarizing humps when depolarized from hyperpolarized potentials. In contrast to PV-positive interneurons, excitatory inputs onto Martinotti cells are strongly facilitating.

The third group of GABAergic cortical interneurons is designated as the 5HT3aR interneuron group. VIP-expressing interneurons are localized in cortical layers II and III. VIP interneurons generally make synapses onto dendrites, and some have been observed to target other interneurons. Relative to all cortical interneurons, VIP interneurons possess a very high input resistance. In general they possess a bipolar, bitufted and multipolar morphology. Irregular spiking interneurons possess a vertically oriented, descending axon that extends to deeper cortical layers, and have an irregular firing pattern that is characterized by action potentials occurring irregularly during depolarizations near threshold, and express the calcium-binding protein calretinin (CR). Other subtypes include rapid-adapting, fast-adapting neurons IS2, as well as a minor population of VIP-positive basket cells with regular, bursting, or irregular-spiking firing patterns. Of the VIP-negative 5HT3aR group, nearly 80% express the interneuron marker Reelin. Neurogliaform cells are a type of cortical interneuron that belongs to this category: they are also known as spiderweb cells and express neuropeptide Y (NPY), with multiple dendrites radiating from a round soma.

Glutamatergic neurons. The mature cerebral cortex harbors a heterogeneous population of glutamatergic neurons, organized into a highly intricate histological architecture. So-called excitatory neurons are usually classified according to the lamina where their soma is located, specific combinations of gene expression, by dendritic morphologies, electrophysiological properties, etc.

Based on the differences in connections, pyramidal neurons are classified as projection neurons with long axons that connect different cortical regions or project to subcortical targets. Cortical projection neurons can be further classified by hodology in associative, commissural and corticofugal subtypes. Associative projection neurons extend axons within a single hemisphere, whereas commissural projection neurons connect neurons in the two cortical hemispheres either through the corpus callosum or the anterior commissure. Corticofugal projection neurons send axons to target areas outside the cerebral cortex, such as the thalamus (corticothalamic neurons), pons (corticopontine neurons (CPN), spinal cord (costicospinal neurons), superior colliculus (corticotectal neurons) and striatum (corticostriatal neurons).

The terms “astrocytic cell,” “astrocyte,” etc. encompass cells of the astrocyte lineage, i.e. glial progenitor cells, astrocyte precursor cells, and mature astrocytes, which for the purposes of the present invention arise from a non-astrocytic cells (i.e., glial progenitors). Astrocytes can be identified by markers specific for cells of the astrocyte lineage, e.g. GFAP, ALDH1L1, AQP4, EAAT1 and EAAT2, etc. Markers of reactive astrocytes include S100, VIM, LCN2, FGFR3 and the like. Astrocytes may have characteristics of functional astrocytes, that is, they may have the capacity of promoting synaptogenesis in primary neuronal cultures; of accumulating glycogen granules in processes; of phagocytosing synapses; and the like. An “astrocyte precursor” is defined as a cell that is capable of giving rise to progeny that include astrocytes.

Astrocytes are the most numerous and diverse neuroglial cells in the CNS. An archetypal morphological feature of astrocytes is their expression of intermediate filaments, which form the cytoskeleton. The main types of astroglial intermediate filament proteins are glial fibrillary acidic protein (GFAP) and vimentin; expression of GFAP, ALDH1L1 and/or AQP4P are commonly used as a specific marker for the identification of astrocytes.

The terms “oligodendrocyte,” “oligodendrocyte progenitor cell,” etc. can encompass cells of the oligodendrocyte lineage, i.e. neural progenitor cells that ultimately give rise to oligodendrocytes, oligodendrocyte precursor cells, and mature and myelinating oligodendrocytes, which for the purposes of the present invention arise from a non-oligodendrocyte cell by experimental manipulation. Oligodendrocytes can be identified by markers specific for cells of the oligodendrocyte lineage as discussed below. Oligodendrocytes may have functional characteristics, that is, they may have the capacity of myelinating neurons; and the like. An “oligodendrocyte precursor” or “oligodendrocyte progenitor cell” is defined as a cell that is capable of giving rise to progeny that include oligodendrocytes. Oligodendrocytes are present in the assembled spheroids.

Oligodendrocytes are the myelin-forming cells of the central nervous system. An oligodendrocyte extends many processes which contact and repeatedly envelope stretches of axons. Subsequent condensation of these wrapped layers of oligodendrocyte membrane form the myelin sheath. One axon may contain myelin segments from many different oligodendrocytes.

Calcium sensors. Neural activity causes rapid changes in intracellular free calcium, which can be used to track the activity of neuronal populations. Art-recognized sensors for this purpose include fluorescent proteins that fluoresce in the presence of changes in calcium concentrations. These proteins can be introduced into cells, e.g. hiPSC, by including the coding sequence on a suitable expression vector, e.g. a viral vector, to genetically modify neurons generated by the methods described herein. GCaMPs are widely used protein calcium sensors, which are comprised of a fluorescent protein, e.g. GFP, the calcium-binding protein calmodulin (CaM), and CaM-interacting M13 peptide, although a variety of other sensors are also available. Many different proteins are available, including, for example, those described in Zhao et al. (2011) Science 333:1888-1891; Mank et al. (2008) Nat. Methods 5(9):805-11; Akerboom et al. (2012) J. Neurosci. 32(40):13819-40; Chen et al. (2013) Nature 499(7458):295-300; etc.; and as described in U.S. Pat. Nos. 8,629,256, 9,518,980 and 9,488,642 and 9,945,844.

Optogenetics integrates optics and genetic engineering to measure and manipulate neurons. Actuators are genetically-encoded tools for light-activated control of proteins; e.g., opsins and optical switches. Opsins are light-gated ion channels or pumps that absorb light at specific wavelengths. Opsins can be targeted and expressed in specific subsets of neurons, allowing precise spatiotemporal control of these neurons by turning on and off the light source. Channel rhodopsins typically allow the fast depolarization of neurons upon exposure to light through direct stimulation of ion channels. Chlamydomonas reinhardtii Channelrhodopsin-1 (ChR1) is excited by blue light and permits nonspecific cation influx into the cell when stimulated. Examples of ChRs from other species include: CsChR (from Chloromonas subdivisa), CoChR (from Chloromonas oogama), and SdChR (from Scherffelia dubia). Synthetic variants have been created, for example ChR2(H134R), C1V1(t/t), ChIEF; ChETA, VChR1, Chrimson, ChrimsonR, Chronos, PsChR2, CoChR, CsChR, CheRiff, and the like. Alternatively, ChR variants that inhibit neurons have been created and identified, for example GtACR1 and GtACR2 (from the cryptophyte Guillardia theta), and variants such as iChloC, SwiChRca, Phobos, Aurora. Halorhodopsin, known as NpHR (from Natronomonas pharaoni), causes hyperpolarization of the cell when triggered with yellow light, variants include Halo, eNpHR, eNpHR2.0, eNpHR3.0, Jaws. Archaerhodopsin-3 (Arch) from Halorubrum sodomense is also used to inhibit neurons.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

### Methods of the Invention

Compositions and methods are provided for in vitro generation of functional human cortico-striatal-midbrain-thalamic-cortical assembled spheroids, which may be generated at least in part from human pluripotent stem cells (hPSCs). Complete cortico-striatal-midbrain-thalamic-cortical spheroids (CSMTC) are assembled from component cultured cell systems, where each cultured cell system is designed to provide specific sets of neural and/or muscle cells, and which components are functionally integrated in the assembled spheroid. Functionally integrated cells interact in a physiologically relevant manner, e.g. forming synapses or neuromuscular junctions, transmitting signals, inducing muscle contractions, forming multicellular structures, and the like.

The methods comprise an initial step of differentiating pluripotent cells, including without limitation induced human pluripotent stem cells (hiPSC), into the component structures of (i) human cortical spheroids (hCS), (ii) human striatum spheroids (hStrS), (iii) human midbrain spheroids (hMbS) and (iv) human diencephalic spheroids (hDiS), and their integration into assembloids. The spheroids may also comprise neural progenitor cells, astrocytes, oligodendrocytes and the like.

The component spheroids are differentiated separately and later assembled by placing them in close proximity with each other for 3 days in an incubator; e.g. (hMbS-hStrS), hMbS and hStrS are generated separately. To generate cortico-striatal-diencephalic assembloids, cortical, striatal and diencephalic spheroids are placed in low attachment plate in an incubator, where they are assembled after 3 days. To generate cortico-striatal-midbrain-thalamic-cortical (CSMTC) or loop assembloids, thalamo-cortical assembloids and meso-striatal assembloids are separately generated, placed in a well of low attachment dish in an incubator, and where they are assembled after 3 days. In some embodiments the neural cells are differentiated from induced human pluripotent stem cells (hiPSC). In some embodiments the hiPSC are derived from somatic cells obtained from neurologically normal individuals. In other embodiments the hiPSC are derived from somatic cells obtained from an individual comprising at least one allele encoding a mutation associated with a neural disease.

Generation of the component spheroids and cells comprised therein utilizes a multi-step process. Initially, hiPSC can be obtained from any convenient source, or can be generated from somatic cells using art-recognized methods. The hiPSC are dissociated from feeders (or if grown in feeder free, aggregated in spheroids of specific sizes) and grown in suspension culture in the absence of FGF2, preferably when dissociated as intact colonies. In certain embodiments the culture is feeder layer free, e.g. when grown on vitronectin coated vessels. The culture may further be free of non-human protein components, i.e. xeno-free, where the term has its usual art-recognized definition, for example referring to culture medium that is free of non-human serum.

The hiPSCs may be cultured in any medium suitable for the growth and expansion of hiPSCs. For example, the medium may be Essential 8 medium. Suspension growth optionally includes in the culture medium an effective dose of a selective Rho-associated kinase (ROCK) inhibitor for the initial period of culture, for up to about 6 hours, about 12 hours, about 18 hours, about 24 hours, about 36 hours, about 48 hours, (see, for example, Watanabe et al. (2007) Nature Biotechnology 25:681 686). Inhibitors useful for such purpose include, without limitation, Rho Kinase Inhibitor IV (Dihydrochloride), Y-27632; Thiazovivin (Cell Res, 2013, 23(10):1187-200; Fasudil (HA-1077) HCl (J Clin Invest, 2014, 124(9):3757-66); GSK429286A (Proc Natl Acad Sci USA, 2014, 111(12):E1140-8); RKI-1447; AT13148; etc. In particular embodiments the ROCK inhibitor Y-27632 is used. Optionally a WNT pathway inhibitor is added. WNT pathway inhibitors that find use in the present disclosure include, without limitation, XAV-939, KY02111, IWR-1, IWP-4, IWP-3, IWP-2, etc.

After about 5 days, about 6 days, about 7 days, about 8 days, about 9 days, about 10 days in suspension culture, the floating spheroids are moved to neural media to differentiate neural progenitors. The media is supplemented with an effective dose of FGF2 and EGF. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

To promote differentiation of neural progenitors into neurons, after about 1 week, about 2 weeks, about 3 weeks, about 4 weeks after FGF2/EGF exposure the neural medium is changed to replace the FGF2 and EGF with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

Human striatal spheroids (hStrS) may be generated by the methods previously described, for example in Miura. Y et al. (2020) Nat Biotechnol.Dec.; 38(12):1421-1430, entitled “Generation of human striatal organoids and cortico-striatal assembloids from human pluripotent stem cells” and in U.S. patent application Ser. No. 17/773,429, herein specifically incorporated by reference.

To generate hStrS, on day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with the Wnt pathway inhibitor IWP-2 (2.5 M, Selleckchem, S7085) and Recombinant Human/Murine/Rat Activin A (50 ng ml-1, PeproTech, 120-14P). On day 11 of differentiation, spheroids were supplemented with the pan retinoid X receptor (RXR) agonist, SR11237 (100 nM, Tocris, 3411), in addition to the compounds described above. From day 23, to promote differentiation of the neural progenitors into neurons, the neural medium was supplemented with brain-derived neurotrophic factor (BDNF; 20 ng ml⁻¹, PeproTech, 450-02), NT3 (20 ng ml⁻¹, PeproTech, 450-03), L-Ascorbic Acid 2-phosphate Trisodium Salt (AA; 200 μM, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534), and 2.5 μM DAPT (STEMCELL Technologies, 72082). From day 43, only neural medium containing B-27™ Supplement, minus vitamin A (Thermo Fisher Scientific, 12587010) or B-27™ Plus Supplement (Thermo Fisher Scientific, A3582801) was used for medium changes every 4 days.

Human midbrain spheroid (hMbS) (also named midbrain organoids) may be generated by the methods previously described, for example in in U.S. patent application Ser. No. 17/773,429, herein specifically incorporated by reference.

To generate hMbS, on day 3 in suspension the spheroids were supplemented with 100 ng/ml FGF8 (PeproTech, 100-25-100 μg) and 1 μM SAG (Millipore Sigma, 566660-1MG), in addition with two SMAD pathway inhibitors. For the first 5 d, Essential 6 medium was changed every day. On day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with the 100 ng/ml FGF8 (day 7-21), 1 μM SAG (day 7-21), 100 nM LDN (day 7-16, Selleckchem, S7507) and 3 μM CHIR (day 8-23). From day 23, to promote differentiation of the neural progenitors into neurons, the neural medium was supplemented with brain-derived neurotrophic factor (BDNF; 20 ng ml⁻¹, PeproTech, 450-02), NT3 (20 ng ml⁻¹, PeproTech, 450-03), L-Ascorbic Acid 2-phosphate Trisodium Salt (AA; 200 μM, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534), and 2.5 μM DAPT (STEMCELL Technologies, 72082). From day 43, neural medium containing B-27™ Plus Supplement (Thermo Fisher Scientific, A3582801) containing L-Ascorbic Acid 2-phosphate Trisodium Salt (AA; 200 μM, Wako, 323-44822) was used for medium changes every 4 days.

Human diencephalic spheroids. To generate hDiS, on day 3 in suspension the spheroids were supplemented with 1 μM CHIR (Selleckchem, S1263), in addition with two SMAD pathway inhibitors. For the first 5 d, medium was changed every day. On day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with 1 μM CHIR (Selleckchem, S1263). On day 9 of differentiation, spheroids were supplemented with 100 nM SAG (Millipore Sigma, 566660-1MG), in addition to the compounds described above. Furthermore, on day 12 of differentiation, spheroids were supplemented with 30 ng/ml BMP7 (PeproTech, 120-03P), in addition to the compounds described above. From day 19, to promote differentiation of the neural progenitors into neurons of hDiS, the neural medium was supplemented with brain-derived neurotrophic factor (BDNF; 20 ng ml⁻¹, PeproTech, 450-02), NT3 (20 ng ml⁻¹, PeproTech, 450-03), L-Ascorbic Acid 2-phosphate Trisodium Salt (AA; 200 μM, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534), and 2.5 μM DAPT (only from day 19-25, STEMCELL Technologies, 72082). From day 43, only neural medium containing B-27™ Plus Supplement (Thermo Fisher Scientific, A3582801) was used for medium changes every 4 days.

In some embodiments, the hDiS comprise glutamatergic neurons expressing TCF7L2. In some embodiments, the hDiS comprise glutamatergic neurons expressing SLC17A6. In some embodiments, the hDiS comprise glutamatergic neurons expressing the thalamus-related gene TCF7L2 and SLC17A6.

Human cortical spheroids. The hCS may be generated by the methods previously described, for example in Pasca et al. (2015) Nat. Methods 12(7):671-678, entitled “Functional cortical neurons and astrocytes from human pluripotent stem cells in 3D culture” and U.S. Pat. No. 10,494,602, herein specifically incorporated by reference.

For example, a suspension culture of hiPS cells is cultured to provide a neural progenitor spheroid, as described above. After about 5 days, about 6 days, about 7 days, about 8 days, about 9 days, about 10 days in suspension culture, the floating neural progenitor spheroids are moved to neural media to differentiate the neural progenitors. The media is supplemented with an effective dose of FGF2 and EGF. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

To promote differentiation of neural progenitors into hCS, comprising glutamatergic neurons, after about 1 week, about 2 weeks, about 3 weeks, about 4 weeks after FGF2/EGF exposure the neural medium is changed to replace the FGF2 and EGF with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml. The cortical spheroids comprise functional glutamatergic neurons.

Following differentiation, the spheroids may then be maintained in culture in neural medium supplemented with BDNF at a concentration of from about 1 to 50 ng/ml, from about 2.5 to 25 ng/ml and may be about 20 ng/ml; IGF at a concentration of from about 1 to 50 ng/ml, from about 2.5 to 25 ng/ml and may be about 10 ng/ml, L-ascorbic acid at a concentration of from about 10 to 500 nM, from about 50 to 250 nM, and may be about 200 nM; and cAMP at a concentration of from about 10 to 500 nM, from about 50 to 150 nM, and may be about 62.5 nM. After such culture, the spheroids can be maintained for extended periods of time in neural medium in the absence of growth factors, e.g. for periods of 1, 2, 3, 4, 5, 6, 7, 8, 9, 10, 11, 12, 24, 36 months or longer.

For example, a suspension culture of hiPSC is induced to a neural fate. For neural induction, an effective dose of an inhibitor of BMP, and of TGFβ pathways is added to the medium, for a period at least about 2 days, at least about 3 days, at least about 4 days, at least about 5 days, and up to about 10 days, up to about 9 days, up to about 8 days, up to about 7 days, up to about 6 days, up to about 5 days. For example, dorsomorphin (DM) or LDN 193189 can be added at an effective dose of at least about 0.1 μM, at least about 1 μM, at least about 5 μM, at least about 10 μM, at least about 50 M, up to about 100 μM concentration, which inhibits bone morphogenetic protein (BMP) type I receptors (ALK2, ALK3 and ALK6). Other useful BMP inhibitors include, without limitation, A 83-01; LDN-193189 (J Clin Invest, 2015, 125(2):796-808); Galunisertib (LY2157299) (Cancer Res, 2014, 74(21): 5963-77); LY2109761 (Toxicology, 2014, 326C:9-17); SB525334 (Cell Signal, 2014, 26(12):3027-35); SD-208; EW-7197; Kartogenin; DMH1; LDN-212854; ML347; LDN-193189 HCl (Proc Natl Acad Sci USA, 2013, 110(52): E5039-48); SB505124; Pirfenidone (Histochem Cell Biol, 2014, 10.1007/s00418-014-1223-0); RepSox; K02288; Hesperetin; GW788388; LY364947, etc. SB-431542 can be added at an effective dose of at least about 0.1 μM, at least about 1 μM, at least about 5 μM, at least about 10 μM, at least about 50 μM, up to about 100 μM concentration, which inhibits TGFβ signaling but has no effect on BMP signaling. An effective dose of a wnt inhibitor may be included in the culture medium, for example at a concentration of from about 0.1 μM to about 100 μM, and may be from about 1 μM to about 25 μM, depending on the activity of the inhibitor that is selected.

The spheroids are co-cultured in medium comprising BDNF, NT3, L-ascorbic acid and CAMP at the concentrations disclosed above. Assembly may be performed with spheroids after around about 30 days, about 60 days, about 90 days, about 120 days, about 150 days, about 180 days, about 210 of culture for the hCS spheroids; and after about 15 days, after about 25 days, after about 35 days, after about 45 days after about 50 days of culture.

Human ventral forebrain (sub-pallial) organoids (hSS). Human ventral forebrain organoids may be generated by the methods previously described, for example in Birey F. et al. (2017) Nature. May 4; 545(7652):54-59, entitled “Assembly of functionally integrated human forebrain organoids” and U.S. Pat. No. 10,676,715, each herein specifically incorporated by reference.

The suspension culture of hiPSC is then induced to a neural fate. This culture may be feeder-free and xeno-free. For hSS neural induction, an effective dose of an inhibitor of BMP, and of TGF□ pathways is added to the medium, for a period at least about 2 days, at least about 3 days, at least about 4 days, at least about 5 days, and up to about 10 days, up to about 9 days, up to about 8 days, up to about 7 days, up to about 6 days, up to about 5 days. For example, dorsomorphin (DM) can be added at an effective dose of at least about 0.1 μM, at least about 1 μM, at least about 5 μM, at least about 10 μM, at least about 50 μM, up to about 100 μM concentration, which inhibits bone morphogenetic protein (BMP) type I receptors (ALK2, ALK3 and ALK6). Other useful BMP inhibitors include, without limitation, A 83-01; DMH-1; K 02288; ML 347; SB 505124; etc. SB-431542 can be added at an effective dose of at least about 0.1 μM, at least about 1 μM, at least about 5 μM, at least about 10 μM, at least about 50 μM, up to about 100 μM concentration, which inhibits TGFβ signaling but has no effect on BMP signaling. Other useful inhibitors of TGFβ include, without limitation, LDN-193189 (J Clin Invest, 2015, 125(2):796-808); Galunisertib (LY2157299) (Cancer Res, 2014, 74(21):5963-77); LY2109761 (Toxicology, 2014, 326C:9-17); SB525334 (Cell Signal, 2014, 26(12):3027-35); SD-208; EW-7197; Kartogenin; DMH1; LDN-212854; ML347; LDN-193189 HCl (Proc Natl Acad Sci USA, 2013, 110(52): E5039-48); SB505124; Pirfenidone (Histochem Cell Biol, 2014, 10.1007/s00418-014-1223-0); RepSox; K02288; Hesperetin; GW788388; LY364947, A 83-01, etc.

Early organoids patterned by SMAD inhibition, e.g. at the time of transfer to the SMAD inhibitory medium, after about 12 hours, after about 24 hours, after about 1 day, after about 2 days, after about 3 days, after about 4 days, are cultured in the presence of an effective dose of a Wnt inhibitor and an SHH inhibitor in the culture medium. The Wnt and SHH inhibitors are maintained for a period of about 7 days, about 10 days, about 14 days, about 18 days, about 21 days, about 24 days, for example at a concentration of from about 0.1 μM to about 100 μM, and may be from about 1 μM to about 50 μM, from about 5 μM to about 25 μM, etc. depending on the activity of the inhibitor that is selected.

Exemplary WNT inhibitors include, without limitation, XAV-939 selectively inhibits Wnt/B-catenin-mediated transcription through tankyrase1/2 inhibition with IC50 of 11 nM/4 nM in cell-free assays; ICG-001 antagonizes Wnt/B-catenin/TCF-mediated transcription and specifically binds to element-binding protein (CBP) with IC50 of 3 μM; IWR-1-endo is a Wnt pathway inhibitor with IC50 of 180 nM in L-cells expressing Wnt3A, induces Axin2 protein levels and promotes B-catenin phosphorylation by stabilizing Axin-scaffolded destruction complexes; Wnt-C59 (C59) is a PORCN inhibitor for Wnt3A-mediated activation of a multimerized TCF-binding site driving luciferase with IC50 of 74 pM in HEK293 cells; LGK-974 is a potent and specific PORCN inhibitor, and inhibits Wnt signaling with IC50 of 0.4 nM in TM3 cells; KY02111 promotes differentiation of hPSCs to cardiomyocytes by inhibiting Wnt signaling, may act downstream of APC and GSK3B; IWP-2 is an inhibitor of Wnt processing and secretion with IC50 of 27 nM in a cell-free assay, selective blockage of Porcn-mediated Wnt palmitoylation, does not affect Wnt/B-catenin in general and displays no effect against Wnt-stimulated cellular responses; IWP-L6 is a highly potent Porcn inhibitor with EC50 of 0.5 nM; WIKI4 is a novel Tankyrase inhibitor with IC50 of 15 nM for TNKS2, and leads to inhibition of Wnt/beta-catenin signaling; FH535 is a Wnt/B-catenin signaling inhibitor and also a dual PPARγ and PPARδ antagonist.

SHH agonists include smoothened agonist, SAG, CAS 364590-63-6, which modulates the coupling of Smo with its downstream effector by interacting with the Smo heptahelical domain (K_{D}=59 nM). SAG may be provided in the medium at a concentration of from about 10 nM to about 10 μM, from about 50 nM to about 1 μM, from about 75 nM to about 500 nM, and may be around about 100 nM.

Optionally the medium in this stage of the hSS culture process further comprises allopregnenolone from about day 10 to about day 23, e.g. from day 11, 12, 13, 14, 15, 16, 17, 18, 19, 20, 21, 22 until the conclusion of the stage; at a concentration of from about 10 nM to about 10 μM, from about 50 nM to about 1 μM, from about 75 nM to about 500 nM, and may be around about 100 nM.

Optionally the hSS cultures are transiently exposed to retinoic acid, e.g. for about 1 to about 4 days, which may be from about day 10 to about day 20, from about day 12 to about day 15, etc., at a concentration of from about 10 nM to about 10 μM, from about 50 nM to about 1 μM, from about 75 nM to about 500 nM, and may be around about 100 nM.

For hSS conditions, after about 5 days, about 6 days, about 7 days, about 8 days, about 9 days, about 10, after about 15 days, after about 20 days, after about 25 days, e.g. around about 23 days, in suspension culture, the floating organoids are moved to neural media to differentiate neural progenitors. The media is supplemented with an effective dose of FGF2 and EGF. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

To promote differentiation of neural progenitors into neurons, after about 1 week, about 2 weeks, about 3 weeks, about 4 weeks after FGF2/EGF exposure the neural medium is changed to replace the FGF2 and EGF with an effective dose of BDNF and NT3. The growth factors can be provided at a concentration for each of at least about 0.5 ng/ml, at least about 1 ng/ml, at least about 5 ng/ml, at least about 10 ng/ml, at least about 20 ng/ml, up to about 500 ng/ml, up to about 250 ng/ml, up to about 100 ng/ml.

After about 4 weeks, about 5 weeks, about 6 weeks, about 7 weeks after FGF2/EGF exposure, the spheroids can be maintained for extended periods of time in neural medium in the absence of growth factors, e.g. for periods of 1, 2, 3, 4, 5, 6, 7, 8, 9, 10, 11, 12 months or longer. The number of astrocytes in the cultures are initially low for the first month, and increase in number after that, up to from about 5%, about 10%, about 15%, about 20%, about 25%, to about 30% or more of the cells in the organoids.

Assembled Spheroids. The individual spheroids can be functionally integrated with separately cultured spheroids, to form a functional human cortico-striatal-midbrain-thalamic-cortical (CSMTC) loop circuit. Complete cortico-striatal-midbrain-thalamic-cortical spheroids (CSMTC) are assembled from component cultured cell systems. Each of human cortical spheroids (hCS); human diencephalic spheroids (hDiS); human midbrain spheroids (hMbS) and human striatal spheroids (hStrS) are separately differentiated, and then integrates in a desired combination (three-parts, four-parts) and desired spatial arrangements (linear, circular) that allow formation of reciprocal connections and enable loop circuit-like properties. A three-part assembled spheroid created by integrating hCS, hStrS and hDiS has revealed synchronized activity. A four part assembled spheroid from hCS, hDiS, hStrS, hMbS has been successfully maintained in culture for up to 3 months, and may be longer, and can provide a cortical loop circuit. The functionally integrated cells interact in a physiologically relevant manner, e.g. forming synapses, transmitting signals, forming multicellular structures, and the like.

The spheroids are co-cultured with each other in neural medium under conditions permissive for cell fusion. Condition permissive for cell fusion may include culturing the spheroids in close proximity, e.g. in direct contact with one another.

Assembly may be performed with spheroids after around about 30 days, about 60 days, about 90 days of culture. The spheroids may be co-cultured for a period of 2 days, 3 days, 5 days, 8 days, 10 days, 14 days, 18 days, 21 days or more. Assembly may be carried out in neural medium. The resulting assembloids are demonstrated to contain functional neural circuits. Methods for confirming the functionality of the neurons are known in the art and include optogenetic methods and imaging of calcium activity in neurons, such as those methods described in the examples. In some embodiments, the methods may comprise confirming the functionality of the neurons in the cortico-raphe nuclei assembloid.

The spheroids may be arranged in any spatial arrangement deeded useful and may be particularly based on the number of distinct spheroids placed together in an assembloid. In some embodiments, when three part assembloids are used the spheroids in the assembloid may be arranged linearly. In some embodiments, when three part assembloids are used the spheroids in the assembloid may be arranged triangularly. In some embodiments, when three part assembloids are used the spheroids in the assembloid may be arranged circularly. In some embodiments, when four part assembloids are used the spheroids in the assembloid may be arranged linearly. In some embodiments, when four part assembloids are used the spheroids in the assembloid may be arranged squarely. In some embodiments, when four part assembloids are used the spheroids in the assembloid may be arranged circularly. In some embodiments, three and four part assembloids may be further supplemented with human subpallial spheroids (hSS) to enable cell migration.

### Screening Assays

In screening assays for the small molecules, the effect of adding a candidate agent to functional assembled spheroids, i.e. hCS-hStrS-hDiS and hCS-hDiS-hStrS-hMbS to determine the effect on neuronal projection, migration, synapse formation, neuron al morphology, intrinsic electrophysical properties, gene expression, cell death or survival (for neurodegeneration related assays) etc. in culture is tested with one or a panel of cellular environments, where the cellular environment includes one or more of: electrical stimulation including alterations in ionicity, stimulation with a candidate agent of interest, contact with other cells including without limitation neurons and neural progenitors, contact with infectious agents, e.g. rabies virus, polio virus, Zika virus, and the like, and where cells may vary in genotype, in prior exposure to an environment of interest, in the dose of agent that is provided, etc. Usually at least one control is included, for example a negative control and a positive control. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the altering of the environment is assessed by monitoring multiple output parameters, including morphological, functional and genetic changes.

Examples of analytic methods comprise, for example, assessing the integration of neurons. Synaptic integration of neurons to neurons can be assessed by using array tomography to detect pre- and post-synaptic proteins in hCS before and after fusion, such as the presence of PSDH95 or GPHN, which are postsynaptic proteins. To further examine these synaptic puncta ‘synaptograms’ consisting of a series of high-resolution sections through a single synapse may be obtained. Whole-cell voltage clamp recordings of synaptic responses can be performed on slices on the functional assembled spheroids, and to distinguish between excitatory postsynaptic currents (EPSCs, downward deflecting) and IPSCs (upward deflecting), a low Cl⁻ solution may be used in the patch pipette with cells held at −40 mV.

Synaptic integration may also be assessed using axon tracing. The axons may be traced in any way deemed useful. The axon tracing may be retrograde tracing or anterograde tracing. The axon tracing may be performed with the use of a viruses, protein or small molecule. Non-limiting examples of viruses that facilitate anterograde axon tracing include, without limitation, herpes simplex virus 1 (HSV-1), HSV-1 strain H129, rhabdoviruses, etc. Non-limiting examples of viruses that facilitate retrograde axon tracing include, without limitation, rabies, pseudorabies, glycoprotein, deleted rabies, etc. Non-limiting examples of proteins and small molecules that facilitate anterograde axon tracing include, without limitation, Phaseolus vulgaris-leucoaggultinin, wheat germ agglutin, dextran amines, etc. Non-limiting examples of proteins and small molecules that facilitate retrograde axon tracing include, without limitation, horse radish peroxidase (HRP), wheat germ agglutin, cholera toxin subunit B, hydroxystilbamidine, Fast Blue, Diamidino Yellow, True Blue, the, carbocyanines Dil and DiO, fluorescent lax microspheres, etc. Non-limiting examples of proteins and small molecules that facilitate retrograde axon tracing include, without limitation, horse radish peroxidase (HRP), wheat germ agglutin, cholera toxin subunit B, hydroxystilbamidine, Fast Blue, Diamidino Yellow, True Blue, the, carbocyanines Dil and DiO, fluorescent lax microspheres, etc. Other viruses, proteins and small molecules that facilitate axon tracing have been described in the art, for example, in Xu, X. et al. (Neuron. 2020 Sep. 23; 107(6):1029-1047) and in Saleeba, C. et al. (Front Neurosci. 2019 Aug. 27; 13:897), each of which herein specifically incorporated by reference.

Live imaging of cells may be performed, and cells modified to express a detectable marker. Calcium sensitive dyes can be used, e.g. Fura-2 calcium imaging; Fluo-4 calcium imaging, Cal-590 calcium imaging, GCaMP6 calcium imaging, voltage imaging using voltage indicators such as voltage-sensitive dyes (e.g. di-4-ANEPPS, di-8-ANEPPS, and RH237) and/or genetically-encoded voltage indicators (e.g. ASAP1, Archer) can be used on the intact spheroids, assembled spheroids, or on cells isolated therefrom.

Calcium imaging assays can be used to determine the functional of neuronal circuits by exploiting the fact that neural activity causes rapid changes in intracellular free calcium. This may involve modifying neurons to contain genetically-encoded calcium indicator proteins or using calcium sensitive dyes such as those described above, such proteins include the fluorophore sensor GCaMP and imaging those cells. GCaMP comprises a circularly permuted green fluorescent protein, a calcium-binding protein calmodulin (CaM) and CaM-interacting M13 peptide, where brightness of the GFP increases upon calcium binding. Further details about calcium imaging assays are described in Chen et al. (2013) Nature 499(7458): 295-300. Other calcium imaging assays include Fura-2 calcium imaging; Fluo-4 calcium imaging, and Cal-590 calcium imaging.

For example, the neurons may be modified to express GCaMP6f. This can be combined with methods that activate certain neurons in response to external stimuli, for example optogenetic methods that activate neurons in response to light. For example, to test functionality between two types of neurons involved in a neural circuit, a “first” neuron can be modified to express an optogenetic actuator (e.g. ChrimsonR) and a “second” neuron modified to express a calcium indicator (e.g. GCaMP6f) and imaging used to monitor calcium release. If the first neuron is functionally connected (synapses with) the second neuron then optogenetic activation of the first neuron will elicit calcium release and a visible readout in the second neuron. As set out in U.S. patent Ser. No. 17/773,429, such a method was used to confirm functionality of the cortico-striatal circuits.

Other optogenetic actuators may be used besides ChrimsonR. Optogenetic actuators that find use in the present disclosure include, without limitation, Channelrhodopsin-1 (ChR1), CsChR, CoChR, SdChR, ChR2(H134R), C1V1(t/t), ChIEF; ChETA, VChR1, Chrimson, Chronos, PsChR2, CoChR, CsChR, CheRiff, etc.

Methods of analysis at the single cell level are also of interest, e.g. as described above: live imaging (including confocal or light-sheet microscopy), single cell gene expression or single cell RNA sequencing, calcium imaging, immunocytochemistry, patch-clamping, flow cytometry and the like. Various parameters can be measured to determine the effect of a drug or treatment on the functional assembled spheroids or cells derived therefrom.

Imaging of neurons can also be used to assess the effect of candidate agents on aspects of neuronal morphology. Aspects of neuronal morphology that are of interest in the present disclosure include, without limitation, soma diameter, dendrite number, dendrite length, dendrite density, dendritic spine number, dendritic spine length, dendritic spine density, axon length, etc. Imaging of neurons may be used to measure the aspects of neuronal morphology. The neuronal morphology may be measured in a variety of ways. For example, the measuring includes, without limitation, histologically staining the first human neural tissue, antibody staining the first human neural tissue, expressing a detectably labeled protein in the first human neural tissue, etc.

Histological stains that find use in the present disclosure include, without limitation, H&E staining, Nissl staining, Luxol-fast blue staining, Kluver-Barrera staining, Bodian silver staining, Holzer staining, Gallyas-Braak staining, thionine staining, Weil-Myelin staining, Solochrome staining, Perls staining, Fluoro-Jade staining, Congo Red staining, thioflavine S staining, amino cupric silver staining, Neutral Red Counter staining, cupric silver staining, Campbell-Switzer Alzheimer staining, autometallography staining, etc. Antibody stains that find use in the present disclosure include, without limitation, 4G8, 6E10, Ab1-40, Ab1-42, alpha synuclein, Asyn-pSer129, AT8, BrdU+hematoxylin, calbindin, caspase-3, caspase-9, cathepsin-D, CD68, c-fos, ChAT+Nissl, doublecortin, endoglin, ferritin, GAD-67, GFAP, GFP, HulgG, Iba1, Ki-67, LAMP1, luciferase, MAP-2, MBP, mDectin, NeuN, Nestin, Oligo2, Orexin A, parvalbumin, p-c-jun, P.U.1, RGMa, S830, SMI-71, SMI-99, somatostatin, STEM-101, TDP-43, TH, TMEM119, TPH, etc.

Parameters are quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, biopolymers, e.g. polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorically determined epitopes associated with heterodimeric integrins. A parameter may be detection of a specifically modified protein or oligosaccharide. A parameter may be defined by a specific monoclonal antibody or a ligand or receptor binding determinant.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, New York, (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome, for example using CRISPR mediated genomic engineering (see for example Shmakov et al. (2017) Nature Reviews Microbiology 15:169). Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′—NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs.

Agents are screened for biological activity by adding the agent to at least one and usually a plurality of cells, in one or in a plurality of environmental conditions, e.g. following stimulation with an agonist, following electric or mechanical stimulation, etc. The change in parameter readout in response to the agent is measured, desirably normalized, and the resulting screening results may then be evaluated by comparison to reference screening results, e.g. with cells having other mutations of interest, normal astrocytes, astrocytes derived from other family members, and the like. The reference screening results may include readouts in the presence and absence of different environmental changes, screening results obtained with other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to fluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

Neuronal activity parameters. Of interest for the functional assembled spheroids screening system are parameters related to the electrical properties of the neurons and muscle cells and therefore directly informative about function and activity. Methods to measure activity may sense the occurrence of action potentials (spikes), and contractions, or twitches. The characteristics of the occurrence of a single spike or multiple spikes either in timely clustered groups (bursts) or distributed over longer time (spike train) of a single neuron or a group of neurons indicate neuronal activation patterns and thus reflect functional neuronal properties, which can be described my multiple parameters. Such parameters can be used to quantify and describe changes in neuronal activity in the systems of the invention.

Neuronal activity parameters include, without limitation, total number of spikes (per recording period); mean firing rate (of spikes); inter-spike interval (distance between sequential spikes); total number of bursts (per recording period); burst frequency; number of spikes per burst; burst duration (in milliseconds); inter-burst interval (distance between sequential bursts); burst percentage (the portion of spikes occurring within a burst); total number of network bursts (spontaneous synchronized network activity); network burst frequency; number of spikes per network burst; network burst duration; inter-network-burst interval; inter-spike interval within network bursts; network burst percentage (the portion of bursts occurring within a network burst).

Quantitative readouts of neuronal activity parameters may include baseline measurements in the absence of agents or a pre-defined genetic control condition and test measurements in the presence of a single or multiple agents or a genetic test condition. Furthermore, quantitative readouts of neuronal activity parameters may include long-term recordings and may therefore be used as a function of time (change of parameter value). Readouts may be acquired either spontaneously or in response to or presence of stimulation or perturbation of the complete neuronal network or selected components of the network. The quantitative readouts of neuronal activity parameters may further include a single determined value, the mean or median values of parallel, subsequent or replicate measurements, the variance of the measurements, various normalizations, the cross-correlation between parallel measurements, etc. and every statistic used to a calculate a meaningful and informative factor.

Comprehensive measurements of neuronal activity using electrical or optical recordings of the parameters described herein may include spontaneous activity and activity in response to targeted electrical or optical stimulation, including, for example, ChR2 delivered through lentiviruses, AAVs or pseudo rabies viruses, Neurotransmitter uncaging such glutamate uncaging, GABA uncaging, nicotine uncaging, etc.) of all neuronal cells or a subpopulation of neuronal cells within the integrated spheroids. Furthermore, spontaneous or induced neuronal activity can be measured in the self-assembled functional environment and circuitry of the neural culture or under conditions of selective perturbation or excitation of specific subpopulations of neuronal cells as discussed above.

In the provided assays, comprehensive measurements of neuronal activity can be conducted at different time points along neuronal maturation and usually include a baseline measurement directly before contacting the neural culture with the agents of interest and a subsequent measurement under agent exposure. Moreover, long-term effects of agents on neural maturation and development can be assessed by contacting the immature neural culture at an early time point with agents of interest and acquiring measurements of the same cultures after further maturation at a later time point compared to control cultures without prior agent exposure.

In some embodiments, standard recordings of neuronal activity of mature neural cultures are conducted after about 2 weeks, after about 3 weeks, after about 4 weeks, after about 6 weeks, after about 8 weeks following fusion (i.e. after mixing the different subdomain components of the culture). Recordings of neuronal activity may encompass the measurement of additive, synergistic or opposing effects of agents that are successively applied to the cultures, therefore the duration recording periods can be adjusted according to the specific requirements of the assay. In some embodiments the measurement of neuronal activity is performed for a predetermined concentration of an agent of interest, whereas in other embodiments measurements of neuronal activity can be applied for a range of concentrations of an agent of interest.

In some embodiments the provided assays are used to assess maturation of the neural culture or single components including GABAergic interneurons, glutamatergic neurons, astrocytes, oligodendrocytes, etc. Maturation of neuronal cells can be measured based on morphology, by optically assessing parameters such as neuromuscular junctions, dendritic arborization, axon elongation, total area of neuronal cell bodies, number of primary processes per neuron, total length of processes per neuron, number of branching points per primary process as well as density and size of synaptic puncta stained by synaptic markers such as synapsin-1, synaptophysin, bassoon, PSD95, anti-BTX antibodies (for neuromuscular junctions) and Homer. Moreover, general neuronal maturation and differentiation can be assessed by measuring expression of marker proteins such as MAP2, TUJ-1, NeuN, Tau, PSA-NCAM, and SYN-1 alone or in combination using FACS analysis, immunoblotting, or fluorescence microscopy imaging, patch clamping. Maturation and differentiation of neuronal subtypes can further be tested by measuring expression of specific proteins. For excitatory neuronal cells this includes staining for e.g. VGLUT1/2, GRIA1/2/3/4, GRIN1, GRIN2A/B, GPHN etc. For inhibitory neuronal cells this includes staining for e.g. GABRA2, GABRB1, VGAT, and GAD67. For cholinergic motor neurons this includes staining for e.g. CHAT and VACHT.

The results of an assay can be entered into a data processor to provide a dataset. Algorithms are used for the comparison and analysis of data obtained under different conditions. The effect of factors and agents is read out by determining changes in multiple parameters. The data will include the results from assay combinations with the agent(s), and may also include one or more of the control state, the simulated state, and the results from other assay combinations using other agents or performed under other conditions. For rapid and easy comparisons, the results may be presented visually in a graph, and can include numbers, graphs, color representations, etc.

The dataset is prepared from values obtained by measuring parameters in the presence and absence of different cells, e.g. genetically modified cells, cells cultured in the presence of specific factors or agents that affect neuronal function, as well as comparing the presence of the agent of interest and at least one other state, usually the control state, which may include the state without agent or with a different agent. The parameters include functional states such as synapse formation and calcium ions in response to stimulation, whose levels vary in the presence of the factors. Desirably, the results are normalized against a standard, usually a “control value or state,” to provide a normalized data set. Values obtained from test conditions can be normalized by subtracting the unstimulated control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a dataset may comprise normalized data from one, two or multiple cell types and assay conditions.

The dataset can comprise values of the levels of sets of parameters obtained under different assay combinations. Compilations are developed that provide the values for a sufficient number of alternative assay combinations to allow comparison of values.

A database can be compiled from sets of experiments, for example, a database can contain data obtained from a panel of assay combinations, with multiple different environmental changes, where each change can be a series of related compounds, or compounds representing different classes of molecules.

Mathematical systems can be used to compare datasets, and to provide quantitative measures of similarities and differences between them. For example, the datasets can be analyzed by pattern recognition algorithms or clustering methods (e.g. hierarchical or k-means clustering, etc.) that use statistical analysis (correlation coefficients, etc.) to quantify relatedness. These methods can be modified (by weighting, employing classification strategies, etc.) to optimize the ability of a dataset to discriminate different functional effects. For example, individual parameters can be given more or less weight when analyzing the dataset, in order to enhance the discriminatory ability of the analysis. The effect of altering the weights assigned each parameter is assessed, and an iterative process is used to optimize pathway or cellular function discrimination.

The comparison of a dataset obtained from a test compound, and a reference dataset(s) is accomplished by the use of suitable deduction protocols, Al systems, statistical comparisons, etc. Preferably, the dataset is compared with a database of reference data. Similarity to reference data involving known pathway stimuli or inhibitors can provide an initial indication of the cellular pathways targeted or altered by the test stimulus or agent.

A reference database can be compiled. These databases may include reference data from panels that include known agents or combinations of agents that target specific pathways, as well as references from the analysis of cells treated under environmental conditions in which single or multiple environmental conditions or parameters are removed or specifically altered. Reference data may also be generated from panels containing cells with genetic constructs that selectively target or modulate specific cellular pathways. In this way, a database is developed that can reveal the contributions of individual pathways to a complex response.

The effectiveness of pattern search algorithms in classification can involve the optimization of the number of parameters and assay combinations. The disclosed techniques for selection of parameters provide for computational requirements resulting in physiologically relevant outputs. Moreover, these techniques for pre-filtering data sets (or potential data sets) using cell activity and disease-relevant biological information improve the likelihood that the outputs returned from database searches will be relevant to predicting agent mechanisms and in vivo agent effects.

For the development of an expert system for selection and classification of biologically active drug compounds or other interventions, the following procedures are employed. For every reference and test pattern, typically a data matrix is generated, where each point of the data matrix corresponds to a readout from a parameter, where data for each parameter may come from replicate determinations, e.g. multiple individual cells of the same type. As previously described, a data point may be quantitative, semi-quantitative, or qualitative, depending on the nature of the parameter.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e. data matrices) obtained from multiple repeated experiments. Classification rules are selected as correctly identifying repeated reference patterns and successfully distinguishing distinct reference patterns. Classification rule-learning algorithms may include decision tree methods, statistical methods, naive Bayesian algorithms, and the like.

A knowledge database will be of sufficient complexity to permit novel test data to be effectively identified and classified. Several approaches for generating a sufficiently encompassing set of classification patterns, and sufficiently powerful mathematical/statistical methods for discriminating between them can accomplish this.

The data from cells treated with specific drugs known to interact with particular targets or pathways provide a more detailed set of classification readouts. Data generated from cells that are genetically modified using over-expression techniques and anti-sense techniques, permit testing the influence of individual genes on the phenotype.

A preferred knowledge database contains reference data from optimized panels of cells, environments and parameters. For complex environments, data reflecting small variations in the environment may also be included in the knowledge database, e.g. environments where one or more factors or cell types of interest are excluded or included or quantitatively altered in, for example, concentration or time of exposure, etc.

For further elaboration of general techniques useful in the practice of this invention, the practitioner can refer to standard textbooks and reviews in cell biology, tissue culture, embryology, stem cell biology, human development and neurobiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXPERIMENTAL

### Example 1

Generation of region-specific brain organoids, including human cortical spheroids (hCS), human striatum spheroids (hStrS), human midbrain spheroids (hMbS) and human diencephalic spheroids (hDiS), and their integration into assembloids

Human induced pluripotent stem (hiPS) cells were cultured on vitronectin-coated plates (5 μg ml¹, Thermo Fisher Scientific, A14700) in Essential 8 medium (Thermo Fisher Scientific, A1517001). Cells were passaged every 4 or 5 days with UltraPure™ 0.5 mM EDTA, pH 8.0 (Thermo Fisher Scientific, 15575020). For the generation of 3D neural spheroids, hiPS cells were incubated with Accutase® (Innovative Cell Technologies, AT-104) at 37° C. for 7-10 min and dissociated into single cells. Optionally, 1-2 day before spheroid formation, hiPS cells can be exposed to 1% dimethylsulfoxide (DMSO) (MilliporeSigma, D2650) in Essential 8 medium. To obtain uniformly sized spheroids, AggreWell-800 (STEMCELL Technologies, 34815) containing 300 microwells was used. Approximately 2 or 3×10⁶ single cells were added per AggreWell-800 well in Essential 8 medium supplemented with the ROCK inhibitor Y27632 (10 μM, Selleckchem, S1049), centrifuged at 100 g for 3 min to capture the cells in the microwells, and incubated at 37° C. with 5% CO₂. After 24h, spheroids consisting of approximately 6,666 or 10,000 cells were collected from each microwell by pipetting medium in the well up and down with a cut P1000 pipet tip and transferred into ultra-low attachment plastic dishes (Corning, 3262) in Essential 6 medium (Thermo Fisher Scientific, A1516401) supplemented with two SMAD pathway inhibitors—dorsomorphin (2.5 μM, Sigma-Aldrich, P5499) and SB-431542 (10 μM, R&D Systems, 1614).

To generate hCS, on day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with 20 ng/ml EGF (R&D Systems, 236-EG) and 20 ng/ml FGF2 (R&D Systems, cat. no. 233-FB). From day 15 of differentiation, media can be changed every other day. From day 23, to promote differentiation of the neural progenitors into neurons, the neural medium was supplemented with brain-derived neurotrophic factor (BDNF; 20 ng ml⁻¹, PeproTech, 450-02), NT3 (20 ng ml⁻¹, PeproTech, 450-03), L-Ascorbic Acid 2-phosphate Trisodium Salt (AA; 200 μM, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534). From day 43, only neural medium containing B-27™ Supplement, minus vitamin A (Thermo Fisher Scientific, 12587010) or B-27™ Plus Supplement (Thermo Fisher Scientific, A3582801) was used for medium changes every 4 days.

To generate hStrS, on day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with the Wnt pathway inhibitor IWP-2 (2.5 μM, Selleckchem, S7085) and Recombinant Human/Murine/Rat Activin A (50 ng ml⁻¹, PeproTech, 120-14P). On day 11 of differentiation, spheroids were supplemented with the pan retinoid X receptor (RXR) agonist, SR11237 (100 nM, Tocris, 3411), in addition to the compounds described above. From day 23, to promote differentiation of the neural progenitors into neurons, the neural medium was supplemented with brain-derived neurotrophic factor (BDNF; 20 ng ml⁻¹, PeproTech, 450-02), NT3 (20 ng ml⁻¹, PeproTech, 450-03), L-Ascorbic Acid 2-phosphate Trisodium Salt (AA; 200 μM, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534), and 2.5 μM DAPT (STEMCELL Technologies, 72082). From day 43, only neural medium containing B-27™ Supplement, minus vitamin A (Thermo Fisher Scientific, 12587010) or B-27™ Plus Supplement (Thermo Fisher Scientific, A3582801) was used for medium changes every 4 days.

To generate hMbS, on day 3 in suspension the spheroids were supplemented with 100 ng/ml FGF8 (PeproTech, 100-25-100 μg) and 1 μM SAG (Millipore Sigma, 566660-1MG), in addition with two SMAD pathway inhibitors. For the first 5 d, Essential 6 medium was changed every day. On day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with the 100 ng/ml FGF8 (day 7-21), 1 μM SAG (day 7-21), 100 nM LDN (day 7-16, Selleckchem, S7507) and 3 μM CHIR (day 8-23). From day 23, to promote differentiation of the neural progenitors into neurons, the neural medium was supplemented with brain-derived neurotrophic factor (BDNF; 20 ng ml-1, PeproTech, 450-02), NT3 (20 ng ml-1, PeproTech, 450-03), L-Ascorbic Acid 2-phosphate Trisodium Salt (AA; 200 M, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534), and 2.5 μM DAPT (STEMCELL Technologies, 72082). From day 43, neural medium containing B-27™ Plus Supplement (Thermo Fisher Scientific, A3582801) containing L-Ascorbic Acid 2-phosphate Trisodium Salt (AA; 200 μM, Wako, 323-44822) was used for medium changes every 4 days.

To generate hDiS, on day 3 in suspension the spheroids were supplemented with 1 μM CHIR (Selleckchem, S1263), in addition with two SMAD pathway inhibitors. For the first 5 d, medium was changed every day. On day 6 in suspension the spheroids were transferred to neural medium containing Neurobasal™-A Medium (Thermo Fisher Scientific, 10888022), B-27™ Supplement, minus vitamin A (Thermo Fisher Scientific, 12587010), GlutaMAX™ Supplement (1:100, Thermo Fisher Scientific, 35050079), Penicillin-Streptomycin (1:100, Thermo Fisher Scientific, 15070063), and supplemented with 1 μM CHIR (Selleckchem, S1263). On day 9 of differentiation, spheroids were supplemented with 100 nM SAG (Millipore Sigma, 566660-1MG), in addition to the compounds described above. Furthermore, on day 12 of differentiation, spheroids were supplemented with 30 ng/mL BMP7 (PeproTech, 120-03P), in addition to the compounds described above. From day 19, to promote differentiation of the neural progenitors into neurons of hDiS, the neural medium was supplemented with brain-derived neurotrophic factor (BDNF; 20 ng ml-1, PeproTech, 450-02), NT3 (20 ng ml⁻¹, PeproTech, 450-03), L-Ascorbic Acid 2-phosphate Trisodium Salt (AA; 200 μM, Wako, 323-44822), N6, 2′-O-Dibutyryladenosine 3′, 5′-cyclic monophosphate sodium salt (cAMP; 50 μM, Millipore Sigma, D0627), cis-4, 7, 10, 13, 16, 19-Docosahexaenoic acid (DHA; 10 μM, Millipore Sigma, D2534), and 2.5 μM DAPT (only from day 19-25, STEMCELL Technologies, 72082). From day 43, only neural medium containing B-27™ Plus Supplement (Thermo Fisher Scientific, A3582801) was used for medium changes every 4 days.

To generate thalamo (diencephlic)-cortical assembloids (hDiS-hCS), hDiS and hCS were separately generated from hiPS cells, and later assembled by placing them in close proximity with each other in 1.5 mL microcentrifuge tubes for 3 days in an incubator. Medium was carefully changed on day 2, and on the third day, assembloids were placed in 24-well ultra-low attachment plates in the neural medium described above using a cut P1000 pipette tip. After this, the medium was changed every 4 days. Assembly was performed at day 63 of differentiation into hDiS and hCS.

We have previously shown the assembly of hCS with hStrS. Briefly, hStrS and hCS were generated from hiPS cells separately, and later assembled by placing them in close proximity with each other in 1.5 mL microcentrifuge tubes for 3 days in an incubator. Medium was carefully changed on day 2, and on the third day, assembloids were placed in 24-well or 60 mm ultralow attachment plates in the neural medium described above using a cut P1000 pipette tip. Assembly was performed at days 63 of hStrS and hCS.

To generate midbrain-striatal assembloids (hMbS-hStrS), hMbS and hStrS were generated separately, and later assembled by placing them in close proximity with each other in 1.5 ml microcentrifuge tubes for 3 days in an incubator. Media was carefully changed on day 2, and on the third day, assembloids were placed in 24-well or 60 mm ultralow attachment plates in the neural medium described above using a cut P1000 pipette tip. Assembly was performed at days 25 of hMbS and hStrS.

To generate cortico-striatal-diencephalic assembloids, cortical, striatal and diencephalic spheroids were placed in tilted in a well of 24-well low attachment plate in an incubator, and then they were assembled after 3 days of assembly. Medium was carefully changed on day 4. After this, media was changed every 4 days. Assembly was performed at days 96 and imaging was performed at day 183.

To generate cortico-striatal-midbrain-thalamic-cortical (CSMTC) or loop assembloids, thalamo-cortical assembloids and meso-striatal assembloids were separately generated were placed in tilted in a well of 6-well low attachment dish in an incubator, and then assembled after 3 days of assembly. Assembly was performed at days 67 and imaged at days 75.

Single cell RNA-seq library preparation and data analysis of hDiS. Dissociation of hDiS was performed as described previously (Sloan, S. A. et al. Nat Protoc. 2018 Sep.; 13(9):2062-2085). Briefly, to obtain single cell suspension from hDiS, randomly selected 4-5 spheroids were pooled, and then incubated in 30 U/mL papain enzyme solution (Worthington Biochemical, LS003126) and 0.4% DNase (12,500 U/mL, Worthington Biochemical, LS2007) at 37° C. for 45 minutes. After enzymatic dissociation, spheroids were washed with a solution including protease inhibitor and gently triturated to achieve a single cell suspension. Cells were resuspended in 0.04% BSA/PBS (Millipore-Sigma, B6917-25MG) and filtered through a 70 μm Flowmi Cell Strainer (Bel-Art, H13680-0070), and number of cells were counted. To target 7,000 cells after recovery, approximately 11,600 cells were loaded per lane on a Chromium Single Cell 3′chip (Chromium Next GEM Chip G Single Cell Kit, 10× Genomics, PN-1000127) and cDNA libraries were generated with the Chromium Next GEM Single Cell 3′ GEM, Library & Gel Bead Kit v3.1 (10× Genomics, PN-1000128), according to the manufacturer's instructions. Each library was sequenced using the Illumina NovaSeq S4 2×150 bp by Admera Health. Quality control, UMI counting of Ensembl genes and aggregation of samples were performed by the ‘count’ and ‘aggr’ functions in Cell Ranger software (version, Cellranger-6.0.1). Further downstream analyses were performed using the R package Seurat. Genes on the X or Y chromosome were removed from the count matrix to avoid biases in clustering due to the sex of the hiPS cell line donors. Cells with more than 7,500 or less than 200 detected genes or with mitochondrial content higher than 15% were also excluded. Genes that were not expressed in at least three cells were not included in the analysis. Gene expression was normalized using a global-scaling normalization method (normalization method, ‘LogNormalize’; scale factor, 10,000), and the 2,000 most variable genes were selected (selection method, ‘vst’) and scaled (mean=0 and variance=1 for each gene) before principal component analysis. The top 10 principal components were used for clustering (resolution of 1.0), using the ‘FindNeighbors’ and ‘FindClusters’ functions, and for visualization with UMAP. Clusters were grouped based on the expression of known marker genes. Unbiased spatial mapping of the whole combined cluster was performed using VoxHunt (version, VoxHunt1.0.0). Briefly, the 50 most variable features from the ISH Allen Brain Atlas data of the E15.5 mouse brain were selected, and similarity maps were calculated. These maps were then plotted in the sagittal views. Comparison of to BrainSpan transcriptomic data of micro-dissected human brain tissue at postconceptional weeks (PCW) 15-24 was also performed using VoxHunt with default settings.

Cryoprotection and immunofluorescence staining. hCS and hDiS were fixed in 4% paraformaldehyde (PFA)/phosphate buffered saline (PBS) overnight at 4° C. They were then washed in PBS and transferred to 30% sucrose/PBS for 2-3 days until the spheroids/assembloids sink in the solution. Subsequently, they were rinsed in optimal cutting temperature (OCT) compound (Tissue-Tek OCT Compound 4583, Sakura Finetek) and 30% sucrose/PBS (1:1), and embedded. For immunofluorescence staining, 18 μm-thick sections were cut using a Leica Cryostat (Leica, CM1850). Cryosections were washed with PBS to remove excess OCT on the sections and blocked in 10% Normal Donkey Serum (NDS, Abcam, ab7475), 0.3% Triton X-100 (Millipore Sigma, T9284-100ML), 1% BSA diluted in PBS for 1 h at room temperature. The sections were then incubated overnight at 4° C. with primary antibodies diluted in PBS containing 2% NDS, 0.1% Triton X-100. PBS was used to wash the primary antibodies and the cryosections were incubated with secondary antibodies in PBS with the PBS containing 2% NDS, 0.1% Triton X-100 for 1 h. The following primary antibodies were used for staining: anti-TCF7L2 (rabbit, Cell Signaling Technology, 2569S, 1:200 dilution), anti-GFP (chicken, GeneTex, GTX13970, 1:1000 dilution), anti-VGLUT2 (mouse, MilliporeSigma, MAB5540, 1:200 dilution), anti-mCherry (rabbit, GeneTex, GTX128508, 1:1000 dilution), anti-MAP2 (guinea pig, Synaptic Systems 188 004, 1:2000 dilution). Alexa Fluor dyes (Life Technologies) were used at 1:1,000 dilution, and nuclei were visualized with Hoechst 33258 (Life Technologies, H3549, 10,000 dilution). Cryosections were mounted for microscopy on glass slides using Aquamount (Polysciences, 18606), and imaged on a Leica TCS SP8 confocal microscope. Images were processed in Fiji (NIH) and IMARIS (Oxford Instruments).

Viral labeling and live cell imaging. The viral infection of the 3D neural spheroids was performed as previously described. In brief, spheroids were transferred to a 1.5 mL Eppendorf tube containing 200 μL of culture medium with virus and incubated overnight at 37C, 5% CO₂. Fresh 800 μL of culture media was added to the tube following day and incubated overnight. The next day, spheroids were transferred into fresh culture media in ultra-low attachment plates. For the live cell imaging, the labeled spheroids or assembloids were transferred to a well in a Corning™ 96-Well Half Area High Content Imaging Glass Bottom Microplate (Corning, 4580) in 150 μL of culture media and incubated in an environmentally controlled chamber for 15-30 min before imaging with Leica TCS SP8. The viruses used in this study are: AAV-DJ-hSyn::eYFP (Stanford University Neuroscience Gene Vector and Virus Core, GVVC-AAV-16), AAV-DJ-hSyn::mCherry (Stanford University Neuroscience Gene Vector and Virus Core, GVVC-AAV-17), and AAV1-hSyn-hChR2(H134R)-EYFP (Addgene, 26973-AAV1).

Clearing and 3D staining of diencephalon (thalamic)-cortical assembloids. To optically clear and image thalamo-cortical assembloids, we applied the hydrophilic chemical cocktail-based CUBIC protocol. Diencephalo (thalamic)-cortical assembloids at day 93 were fixed with a 4% PFA/PBS solution at 4° C. overnight. The next day, assembloids were washed twice with PBS and incubated in Tissue-Clearing Reagent CUBIC-L (TCI, T3740) at 37° C. for 2 d. Assembloids were washed three times with PBS, one time with HEPES-TSB solution for 2 h each and then stained with anti-GFP (chicken, GeneTex, GTX13970, 1:500 dilution), anti-mCherry (rabbit, GeneTex, GTX128508, 1:500 dilution), and anti-MAP2 (guinea pig, Synaptic Systems, 188 044, 1:2,000 dilution) antibodies in HEPES-TSB solution at 37° C. for 2 d. Assembloids were subsequently washed one time with 10% Triton X-100 and one time with HEPES-TSB solution for 2 h each and then incubated with secondary antibodies (Alexa Fluor, 1:1,000 dilution) in HEPES-TSB solution at 37° C. for 2 d. Assembloids were washed two times with 10% Triton X-100 for 30 min and one time with PBS for 1 h and then incubated with 1% PFA/PBS solution at room temperature overnight. After washing with PBS for 2 h, assembloids were incubated with Tissue-Clearing Reagent CUBIC-R+(TCI, T3741) at room temperature for 2 d for refractive index matching. CUBIC-cleared assembloids were then transferred into a well of a Corning 96-well microplate (Corning, 4580) in 150 μl of CUBIC-R+ solution and imaged using a ×10 objective on a Leica TCS SP8 confocal microscope.

Projection imaging in intact cortico-striatal assembloids. The reciprocal projection of thalamo-cortical assembloids including hDiS-derived AAV-DJ-hSyn1::eYFP+ cells into hCS, and hCS-derived AAV-DJ-hSyn1::mCherry+ cells into hCS were imaged under environmentally controlled conditions in intact, thalamo-cortical assembloids using Leica TCS SP8 confocal microscope with a motorized stage. Assembloids were transferred to a well in a Corning™ 96-Well Half Area High Content Imaging Glass Bottom Microplate (Corning, 4580) in 150 μL of culture media and incubated in an environmentally controlled chamber for 15-30 min before imaging. Images were taken using a 10× objective lens at a depth of 0-500 μm.

Calcium dye imaging in intact cortico-striatal-thalamic assembloids. Intact cortico-striatal-thalamic assembloid were incubated 5 μM Calbryte (Calbryte™ 520 AM, AAT Bioquest, 20650), and 5% F127 (Pluronic®) F-127, AAT Bioquest, 20053) for 1 hour at 37° C., and following incubation at room temperature for 15 min. Assembloids were then transferred to glass bottom dish with culture medium. Assembloids were imaged under environmentally controlled conditions using a 5× objective lens with a Leica TCS SP8 confocal microscope at a frame rate of 14.7 frames/sec. Acquire images were processed in Fiji (NIH).

### Results

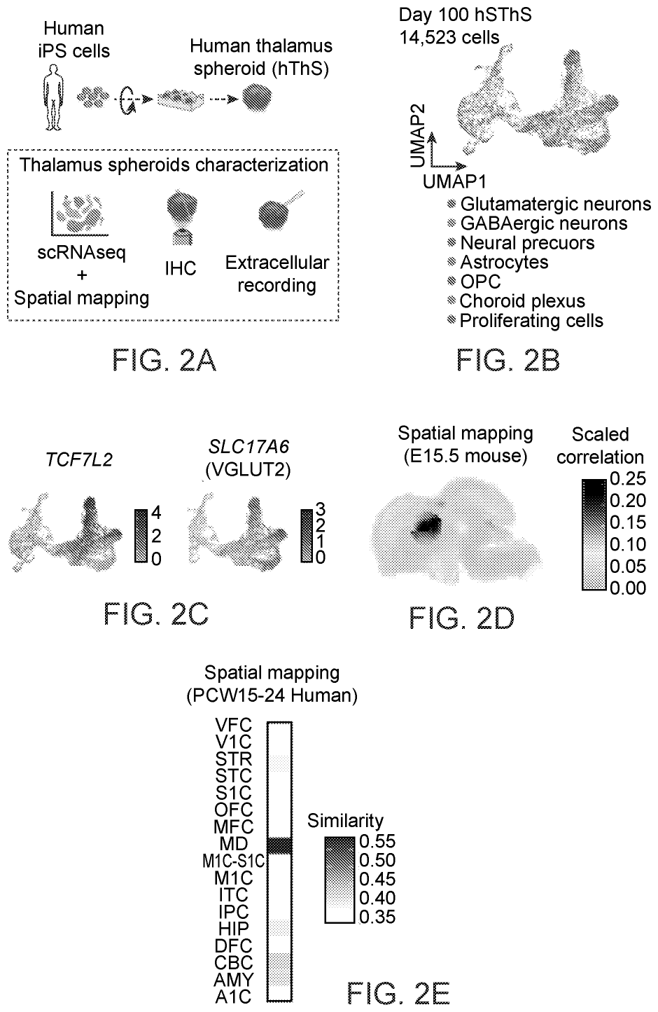

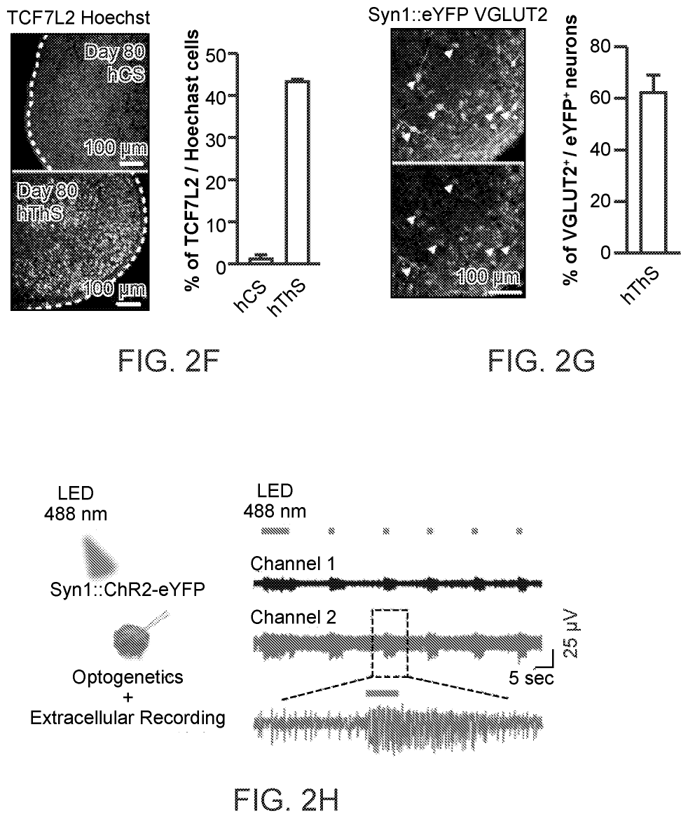

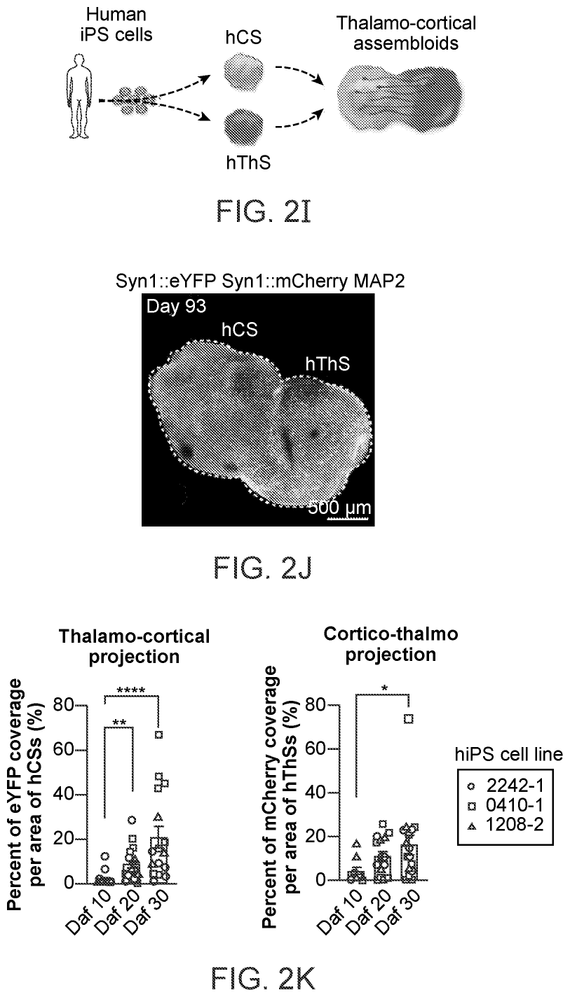

Human diencephalic (thalamo)-cortical assembloids. To generate hDiS resembling the thalamus of the diencephalon, we leveraged approaches to derive region-specific 3D neural cultures that we previously developed (Yoon, S. J. et al. Nat Methods. 2019 Jan.; 16(1):75-78). We enzymatically dissociated hiPS cells into a single cell suspension and aggregated them into spheroids using microwells (FIG. 2A). After dislodging the spheroids from the microwells 18-24 h later, we treated them with the dual SMAD inhibitors dorsomorphin and SB-431542 for 6 days, followed by CHIR (day 3-19), SAG (day 7-19) and BMP7 (day 12-19). To comprehensively study cellular and regional identity of cells in hDiS, we performed droplet-based scRNA-seq analysis at days 100 of in vitro differentiation (n=14,523 cells from three hiPS cell lines). Uniform Manifold Approximation and Projection (UMAP) dimensionality reduction, we identified seven major cell clusters (FIG. 2B), including a major group of glutamatergic neurons expressing the thalamus-related gene TCF7L2 and SLC17A6 (FIG. 2C), a cluster of GABAergic neurons, neural precursors, astrocytes, oligodendrocyte progenitors, choroid plexus, and cycling cells. To verify the cellular and regional identities of cells in hDiS in an unbiased fashion, we mapped the scRNA-seq data onto 3D in situ hybridization data from the Allen Brain Atlas using the VoxHunt algorithm. We found that hDiS cells mapped onto the diencephalon of the embryonic day 15.5 mouse brain (FIG. 2D). Furthermore, we mapped data from hDiS onto the BrainSpan human transcriptomic dataset and found that the highest similarity with the dorsomedial thalamus (DM) at PCW 15-24 (FIG. 2E). Moreover, immunostaining of hDiS at day 80 in vitro differentiation, showed that approximately 40% of cells in hDiS express thalamus-related marker TCF7L2, but not in hCS (FIG. 2F), and approximately 60% of Syn1-labeled neurons in hDiS were glutamatergic VGLUT2+ cells, which is indicative of thalamo-cortical projection neurons (FIG. 2G). To assess functional activity of hDiS neurons, we inserted a high-density electrode array into intact hDiS bathed in artificial cerebrospinal fluid (aCSF) at 37° C., sorted single-units, and observed neurons firing spontaneously at about 1 Hz (FIG. 2H). We also coupled these recordings with optogenetic stimulations and found that activation with blue light of hDiS neurons expressing ChR2-eYFP under Syn1 promoter, triggered high activity firing rates (FIG. 2H). These results indicate the presence of a functional network of thalamic neurons in hDiS.

Thalamo (diencephalic)-cortical assembloids. To develop a model for the thalamo-cortical circuits (FIG. 2I), hDiS and hCS are separately differentiated and placed adjacent to each other inside conical tube, as described previously (Miura, Y. et al. Nat Biotechnol. 2020 Dec.; 38(12):1421-1430). After 3 days, those spheroids were successfully assembled (FIG. 2J) and imaging revealed increase projections in the thalamo-cortical and the cortico-thalamic direction at 20-30 days after fusion (FIG. 2K).

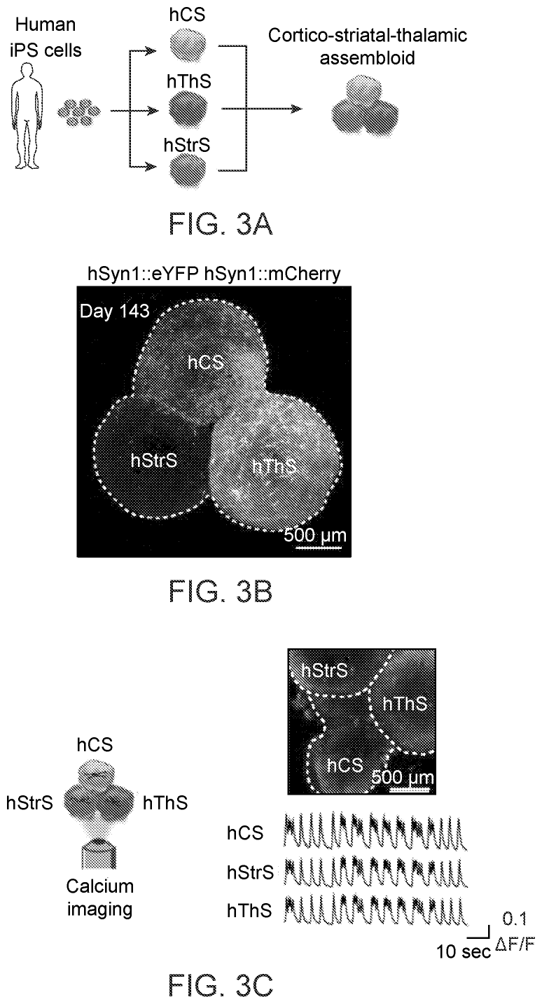

Cortico-striatal-thalamic (diencephalic) assembloids. To form a three-part cortico-striatal-thalamic assembloids, we assembled hDiS, hCS, and hStrS by placing spheroids in close proximity in the shape of triangle (FIG. 3A, B). To study functional connection in the three-part assembloids, we used a green-fluorescent calcium dye and simultaneously imaged neuronal activity in three-part assembloids. We found synchronized calcium activity in the three-part assembloid (FIG. 3C), which is suggestive of functional integration in three-part assembloids.

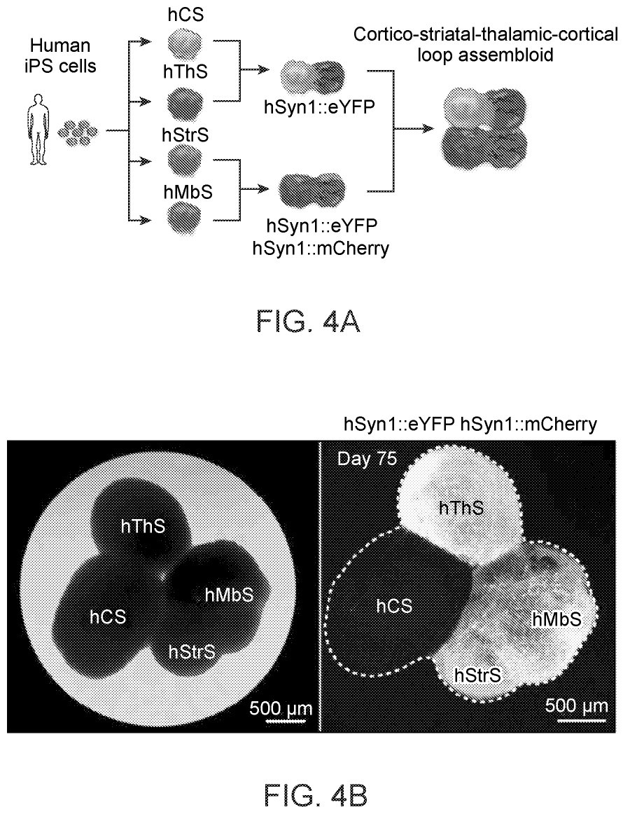

Cortico-striatal-midbrain-thalamic-cortical (CSMTC) loop assembloids. To further develop the cortico-striatal-midbrain-thalamic-cortical (CSMTC) or loop circuits, we leveraged this approach to generate, for the first time, assembloids that include all four parts of the loop: hCS, hDiS, hStrS and hMbS (FIG. 4A, B). hCS-hThS and hStrS-hMbS were generated separately as described above, and then they all assembled to form four-part assembloids in a circle. Fluorescent labeling prior to fusion with AAVs expressing eYFP or mCherry showed that the four organoids have structurally integrated. We have maintained these cultures for at least 3 months. The 4 organoids (spheroids) can also be combined linearly or in other geometrical shapes to study other circuit features. Moreover, interneurons and other cells can be included by fusion of subpallial spheroids (hSS) on the outside of various parts of the loop in a 4+4 arrangement to enable cell migration.

This is the first time where the human cortico-striatal-midbrain-thalamic-cortical (CSMTC) or loop circuits are assembled in vitro using human-derived pluripotent stem cells. Multiple applications can be envisioned using this platform. The novel loop neural circuit assembled system represents a versatile in vitro platform for modeling the formation of neural circuits to study disease-associated cellular phenotypes in axon projection and synapse formation across multiple brain regions, as well as brain region specific cell vulnerability for genetic or environmental effects in health and diseases. This system also has a great potential to be a powerful screening platform for verifying effects of biological manipulations and chemical treatments on neural circuit development of human brain such as various small molecules, drugs, metabolites, microbiome-related compounds, and cytokines. Results from these experiments potentially contribute to the identification of novel therapeutic interventions in the various neurodevelopmental disorders associated with these brain regions.
1 Kuo, H. Y. & Liu, F. C. Synaptic Wiring of Corticostriatal Circuits in Basal Ganglia: Insights into the Pathogenesis of Neuropsychiatric Disorders. eNeuro 6, doi:10.1523/ENEURO.0076-19.2019 (2019).
2 Shepherd, G. M. Corticostriatal connectivity and its role in disease. Nat Rev Neurosci 14, 278-291, doi:10.1038/nrn3469 (2013).
3 Sloan, S. A., Andersen, J., Pasca, A. M., Birey, F. & Pasca, S. P. Generation and assembly of human brain region-specific three-dimensional cultures. Nat Protoc 13, 2062-2085, doi:10.1038/s41596-018-0032-7 (2018).
4 Miura, Y. et al. Generation of human striatal organoids and cortico-striatal assembloids from human pluripotent stem cells. Nat Biotechnol 38, 1421-1430, doi:10.1038/s41587-020-00763-w (2020).
5 Yoon, S. J. et al. Reliability of human cortical organoid generation. Nat Methods 16, 75-78, doi:10.1038/s41592-018-0255-0 (2019).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for producing functionally integrated human cortico-striatal-midbrain-thalamic-cortical assembled spheroids in vitro, the method comprising:
inducing in a pluripotent stem cell suspension culture a neural fate to provide a spheroid of neural progenitor cells wherein the pluripotent stem cell suspension culture is optionally an induced pluripotent stem cell suspension culture;
(i) differentiating the neural progenitor cells in a spheroid to differentiate into one or more of: human cortical spheroids (hCS), human striatum spheroids (hStrS), human midbrain spheroids (hMbS) and human diencephalic spheroids (hDiS); and
culturing the hCS, hStrS, and hDiS or hCS, hDiS, hStrS, and hMbS under conditions permissive for spheroid fusion into loop assembloids while maintaining for an extended period of time in neural medium; wherein an integrated structure is differentiated comprising interacting neurons that form a loop circuit.

2. The method of claim 1, wherein the neurons comprise at least one allele associated with a neurologic disorder.

3. (canceled)

4. The method of claim 1, wherein the pluripotent stem cell suspension culture is induced to a neural fate by culturing intact colonies of the pluripotent stem cells in medium comprising a dose of dorsomorphin (DM) or LDN 193189, and SB-431542 effective to induce pluripotent stem cells to a neural fate.

5. (canceled)

6. The method of claim 1, wherein the suspension culture is feeder layer free.

7. The method of claim 1, wherein cells are differentiated into neural progenitors by culture in neural medium comprising a dose of retinoic acid, FGF2 and EGF.

8. The method of claim 7, further comprising differentiating neural progenitors by supplementing medium with an effective dose of an SHH pathway agonist, optionally supplemented with a gamma secretase inhibitor.

9. The method of claim 1, wherein the spheroids are then cultured in medium comprising an effective dose of BDNF; IGF; L-ascorbic acid; and cAMP to mature spinal cord spheroids.

10. The method of claim 1, further comprising maintaining functionally integrated human hCS-hStrS-hDiS or hCS-hDiS-hStrS-hMbS assembloids thus produced for an extended period of time in neural medium lacking growth factors.

11. The method of claim 1, wherein the hDiS cells express TCF7L2.

12. (canceled)

13. A method determining the effect of a candidate agent on human cortico-striatal-midbrain-thalamic-cortical circuits, the method comprising:
contacting the candidate agent with one or a panel of functionally integrated human hCS-hStrS-hDiS or hCS-hDiS-hStrS-hMbS assembled spheroids differentiated from induced human pluripotent stem cells (hiPSC) according to the method of claim 1, or a population of cells isolated therefrom; and determining the effect of the agent on morphologic, genetic or functional parameters.

14. The method of claim 13, wherein a panel of functionally integrated human cortico-striatal-midbrain-thalamic-cortical assembled spheroids comprises at least 2 differing genotypes.

15. An in vitro generated functionally integrated human c hCS-hStrS-hDiS or hCS-hDiS-hStrS-hMbS assembled spheroid produced by the method of claim 1.

16. A method for producing a human diencephalic spheroid or organoid (hDIS), the method comprising:
(a) inducing a human pluripotent stem cell in suspension culture to a neural fate to provide a neural spheroid wherein the human pluripotent stem cell is optionally an induced human pluripotent stem cell;
(b) differentiating the neural spheroid into a diencephalic spheroid; and
(c) maintaining the diencephalic spheroid in neural medium such that the diencephalic spheroid comprises human diencephalic neurons.

17. The method of claim 16, wherein the human diencephalic neurons comprise glutamatergic neurons expressing TCF7L2 and SLC17A6.

18. (canceled)

19. The method of claim 16, wherein inducing the human pluripotent stem cell in suspension culture to the neural fate in step (a) comprises culturing in a medium comprising one or more SMAD inhibitors, and an inhibitor of GSK-3, BMP7 and a sonic hedgehog pathway agonist.

20. The method of claim 19, wherein the SMAD inhibitors are dorsomorphin (DM) or LDN 193189, and SB-431542, the sonic hedgehog pathway agonist is smoothened agonist (SAG), and the inhibitor of GSK-3 is CHIR99021.

21. (canceled)

22. The method of claim 16 wherein step (a) comprises culturing in a medium comprising the one or more SMAD inhibitors is for a period of from 4 to 10 days, and adding the inhibitor of GSK-3 to the medium after about 3 days of culture in the medium comprising the one or more SMAD inhibitors and adding the sonic hedgehog pathway agonist to the medium after about 7 days of culture in the medium comprising the one or more SMAD inhibitors and adding the BMP7 to the medium after about 12 days of culture in the medium comprising the one or more SMAD inhibitors.

23. The method of claim 16, wherein differentiating the neural spheroid into a diencephalic spheroid in step (b) comprises:
transferring the neural spheroid to a suspension culture in neural medium supplemented with one or more SMAD inhibitors, and an inhibitor of GSK-3, BMP7 and a sonic hedgehog pathway agonist; and
culturing the neural spheroid in suspension culture in neural medium supplemented with brain-derived neurotrophic factor (BDNF), NT3, L-Ascorbic Acid, 5′-cyclic monophosphate sodium salt (cAMP), cis-4, 7, 10, 13, 16, 19-Docosahexaenoic acid (DHA), and DAPT.

24. The method of claim 23, wherein the sonic hedgehog pathway agonist is smoothened agonist (SAG), the inhibitor of SMAD is dorsomorphin (DM) or LDN 193189, and SB-431542, and the inhibitor of GSK-3 is CHIR99021.

25. A diencephalic spheroid obtained by the method of claim 16.
